(19) United States
(12) Patent Application Publication
(10) Pub. No.: US 2008/0292561 A1
(43) Pub. Date: Nov. 27, 2008

(54) Pyrazolo-Pyrimidine Derivatives as Anti-Inflammatory Agents

(75) Inventors: Keith Biggadike, Hertfordshire (GB); David House, Hertfordshire (GB); Graham George Adam Inglis, Hertfordshire (GB); Simon John Fawcett MacDonald, Hertfordshire (GB); Iain McFarlane McLay, Hertfordshire (GB); Philip Alan Skone, Hertfordshire (GB)

Correspondence Address: GLAXOSMITHKLINE;CORPORATE INTELLECTUAL PROPERTY, MAI B482, FIVE MOORE DR., PO BOX 13398, RESEARCH TRIANGLE PARK, NC, 27709-3398, US

(73) Assignee: Glaxo Group Limited

(21) Appl. No.: 12/092,974

(22) PCT Filed: Nov. 7, 2006
(86) PCT No.: PCT/EP2006/010730

§ 371 Date: May 8, 2008

(30) Foreign Application Priority Data
Nov. 9, 2005 (GB) 0522880.4

(51) Int. Cl. A61K 9/12; C07D 487/04; A61P 17/00; A61K 31/519

(52) U.S. Cl. 424/45; 544/262; 514/262.1

(57) ABSTRACT

A compound of formula (I):

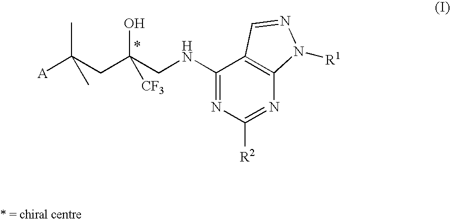

is claimed
wherein
A represents 2,3-dihydro-1-benzofuran-7-yl, 5-fluoro-2-methoxy-phenyl or 5-fluoro-2-hydroxy-phenyl;
R¹ represents phenyl, pyridyl or thienyl wherein the phenyl group may be optionally substituted by one or two groups independently selected from fluorine, cyano, —C(O)OCH₃ and —C(O)OCH₂CH₃, and the pyridyl group may be optionally substituted by one fluorine group; and
R² represents methyl or ethyl; along with physiologically functional derivatives thereof, pharmaceutical compositions and formulations thereof, combinations thereof, methods of treatment employing such compounds and processes for preparing the compounds.

The present invention relates to non-steroidal compounds and a process for their preparation, to pharmaceutical compositions comprising the compounds and the preparation of said compositions, to intermediates and to use of the compounds for the manufacture of a medicament for therapeutic treatment, particularly for the treatment of inflammation and/or allergic conditions.

Nuclear receptors are a class of structurally related proteins involved in the regulation of gene expression. The steroid hormone receptors are a subset of this family whose natural ligands typically comprise endogenous steroids such as estradiol (estrogen receptor), progesterone (progesterone receptor) and cortisol (glucocorticoid receptor). Man-made ligands to these receptors play an important role in human health, in particular the use of glucocorticoid agonists to treat a wide range of inflammatory conditions.

Glucocorticoids exert their actions at the glucocorticoid receptor (GR) through at least two intracellular mechanisms, transactivation and transrepression (see: Schacke, H., Docke, W-D. & Asadullah, K. (2002) Pharmacol and Therapeutics 96:23-43; Ray, A., Siegel, M.D., Prefontaine, K. E. & Ray, P. (1995) Chest 107:139S; and Konig, H., Ponta, H., Rahmsdorf, H. J. & Herrlich, P. (1992) EMBO J 11:2241-2246). Transactivation involves direct binding of the glucocorticoid receptor to distinct deoxyribonucleic acid (DNA) glucocorticoid response elements (GREs) within gene promoters, usually but not always increasing the transcription of the downstream gene product. Recently, it has been shown that the GR can also regulate gene expression through an additional pathway (transrepression) in which the GR does not bind directly to DNA. This mechanism involves interaction of the GR with other transcription factors, in particular NFkB and AP1, leading to inhibition of their pro-transcriptional activity (Schacke, H., Docke, W-D. & Asadullah, K. (2002) Pharmacol and Therapeutics 96:23-43; and Ray, A., Siegel, M. D., Prefontaine, K. E. & Ray, P. (1995) Chest 107:139S). Many of the genes involved in the inflammatory response are transcriptionally activated through the NFkB and AP1 pathways and therefore inhibition of this pathway by glucocorticoids may explain their anti-inflammatory effect (see: Barnes, P. J. & Adcock, I. (1993) Trend Pharmacol Sci 14: 436-441; and Cato, A. C. & Wade, E. (1996) Bioessays 18: 371-378).

Despite the effectiveness of glucocorticoids in treating a wide range of conditions, a number of side-effects are associated with pathological increases in endogenous cortisol or the use of exogenous, and particularly systemically administered, glucocorticoids. These include reduction in bone mineral density (Wong, C. A., Walsh, L. J., Smith, C. J. et al. (2000) Lancet 355:1399-1403), slowing of growth (Allen, D. B. (2000) Allergy 55: suppl 62, 15-18), skin bruising (Pauwels, R. A., Lofdahl, C. G., Latinen, L. A. et al. (1999) N Engl J Med 340:1948-1953), development of cataracts (Cumming, R. G., Mitchell, P. & Leeder, S. R. (1997) N Engl J Med 337:8-14) and dysregulation of lipid and glucose metabolism (Faul, J. L., Tormey, W., Tormey, V. & Burke, C. (1998) BMJ 317:1491; and Andrews, R. C. & Walker, B. R. (1999) Clin Sci 96:513-523). The side-effects are serious enough often to limit the dose of glucocorticoid that can be used to treat the underlying pathology leading to reduced efficacy of treatment.

Current known glucocorticoids have proved useful in the treatment of inflammation, tissue rejection, auto-immunity, various malignancies, such as leukemias and lymphomas, Cushing's syndrome, rheumatic fever, polyarteritis nodosa, granulomatous polyarteritis, inhibition of myeloid cell lines, immune proliferation/apoptosis, HPA axis suppression and regulation, hypercortisolemia, modulation of the Th1/Th2 cytokine balance, chronic kidney disease, stroke and spinal cord injury, hypercalcemia, hyperglycemia, acute adrenal insufficiency, chronic primary adrenal insufficiency, secondary adrenal insufficiency, congenital adrenal hyperplasia, cerebral edema, thrombocytopenia and Little's syndrome.

Glucocorticoids are especially useful in disease states involving systemic inflammation such as inflammatory bowel disease, systemic lupus erythematosus, polyarteritis nodosa, Wegener's granulomatosis, giant cell arteritis, rheumatoid arthritis, osteoarthritis, seasonal rhinitis, allergic rhinitis, vasomotor rhinitis, urticaria, angioneurotic edema, chronic obstructive pulmonary disease, asthma, tendonitis, bursitis, Crohn's disease, ulcerative colitis, autoimmune chronic active hepatitis, organ transplantation, hepatitis and cirrhosis. Glucocorticoids have also been used as immunostimulants and repressors and as wound healing and tissue repair agents.

Glucocorticoids have also found use in the treatment of diseases such as inflammatory scalp alopecia, panniculitis, psoriasis, discoid lupus erythemnatosus, inflamed cysts, atopic dermatitis, pyoderma gangrenosum, pemphigus vulgaris, bullous pemphigoid, systemic lupus erythematosus, dermatomyositis, herpes gestationis, eosinophilic fascitis, relapsing polychondritis, inflammatory vasculitis, sarcoidosis, Sweet's disease, type 1 reactive leprosy, capillary hemangiomas, contact dermatitis, atopic dermatitis, lichen planus, exfoliative dermatitis, erythema nodosum, acne, hirsutism, toxic epidermal necrolysis, erythema multiform and cutaneous T-cell lymphoma.

WO00/32584, WO02/10143, WO03/082827, WO03/082280, DE10261874, WO05/003098 and WO05/030213 disclose certain non-steroidal anti-inflammatory agents.

The present invention provides compounds of formula (I):

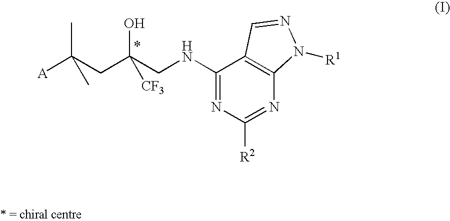

wherein
A represents 2,3-dihydro-1-benzofuran-7-yl, 5-fluoro-2-methoxy-phenyl or 5-fluoro-2-hydroxy-phenyl;
R¹ represents phenyl, pyridyl or thienyl wherein the phenyl group may be optionally substituted by one or two groups independently selected from fluorine, cyano, —C(O)OCH₃ and —C(O)OCH₂CH₃, and the pyridyl group may be optionally substituted by one fluorine group; and
R² represents methyl or ethyl;
and physiologically functional derivatives thereof (hereinafter “compounds of the invention”).

Compounds of formula (I) contain one chiral centre. A single enantiomer or mixture of enantiomers (eg. racemic mixture) may be preferred.

In one embodiment, A represents 2,3-dihydro-1-benzofuran-7-yl. In another embodiment, A represents 5-fluoro-2-methoxy-phenyl. In a further embodiment, A represents 5-fluoro-2-hydroxy-phenyl.

In one embodiment, R¹ represents phenyl optionally substituted by one or two fluorine groups. In another embodiment, R¹ represents 2-fluorophenyl. In a further embodiment R¹ represents 2,4-difluorophenyl.

In one embodiment R¹, represents pyridyl optionally substituted by one fluorine group. In a further embodiment, R¹ represents 4-pyridyl.

In one embodiment, R² represents methyl.

It is to be understood that the present invention encompasses all combinations of the substituent groups described above.

In one embodiment, the compound of formula (I) is:
4-(2,3-dihydro-1-benzofuran-7-yl)-1,1,1-trifluoro-4-methyl-2-{[(6-methyl-1-phenyl-1H-pyrazolo[3,4-d]pyrimidin-4-yl)amino]methyl}-2-pentanol;
4-(2,3-dihydro-1-benzofuran-7-yl)-1,1,1-trifluoro-2-({[1-(2-fluorophenyl)-6-methyl-1H-pyrazolo[3,4-d]pyrimidin-4-yl]amino}methyl)-4-methyl-2-pentanol;
4-(2,3-dihydro-1-benzofuran-7-yl)-1,1,1-trifluoro-2-({[1-(4-fluorophenyl)-6-methyl-1H-pyrazolo[3,4-d]pyrimidin-4-yl]amino}methyl)-4-methyl-2-pentanol;
2-({[1-(2,4-difluorophenyl)-6-methyl-1H-pyrazolo[3,4-d]pyrimidin-4-yl]amino}methyl)-4-(2,3-dihydro-1-benzofuran-7-yl)-1,1,1-trifluoro-4-methyl-2-pentanol;
4-(2,3-dihydro-1-benzofuran-7-yl)-2-{[(6-ethyl-1-phenyl-1H-pyrazolo[3,4-d]pyrimidin-4-yl)amino]methyl}-1,1,1-trifluoro-4-methyl-2-pentanol;
4-(2,3-dihydro-1-benzofuran-7-yl)-2-({[6-ethyl-1-(2-fluorophenyl)-1H-pyrazolo[3,4-d]pyrimidin-4-yl]amino}methyl)-1,1,1-trifluoro-4-methyl-2-pentanol;
4-(2,3-dihydro-1-benzofuran-7-yl)-2-({[6-ethyl-1-(4-fluorophenyl)-1H-pyrazolo[3,4-d]pyrimidin-4-yl]amino}methyl)-1,1,1-trifluoro-4-methyl-2-pentanol;
2-({[1-(2,4-difluorophenyl)-6-ethyl-1H-pyrazolo[3,4-d]pyrimidin-4-yl]amino}methyl)-4-(2,3-dihydro-1-benzofuran-7-yl)-1,1,1-trifluoro-4-methyl-2-pentanol;
4-(2,3-dihydro-1-benzofuran-7-yl)-2-({[6-ethyl-1-(3-pyridinyl)-1H-pyrazolo[3,4-d]pyrimidin-4-yl]amino}methyl)-1,1,1-trifluoro-4-methyl-2-pentanol;
4-(2,3-dihydro-1-benzofuran-7-yl)-2-({[6-ethyl-1-(4-pyridinyl)-1H-pyrazolo[3,4-d]pyrimidin-4-yl]amino}methyl)-1,1,1-trifluoro-4-methyl-2-pentanol;
4-(2,3-dihydro-1-benzofuran-7-yl)-2-({[6-ethyl-1-(6-fluoro-3-pyridinyl)-1H-pyrazolo[3,4-d]pyrimidin-4-yl]amino}methyl)-1,1,1-trifluoro-4-methyl-2-pentanol;
4-(2,3-dihydro-1-benzofuran-7-yl)-2-({[6-ethyl-1-(3-thienyl)-1H-pyrazolo[3,4-d]pyrimidin-4-yl]amino}methyl)-1,1,1-trifluoro-4-methyl-2-pentanol;
1,1,1-trifluoro-4-[5-fluoro-2-(methyloxy)phenyl]-4-methyl-2-{[(6-methyl-1-phenyl-1H-35 pyrazolo[3,4-d]pyrimidin-4-yl)amino]methyl}-2-pentanol;
1,1,1-trifluoro-4-[5-fluoro-2-(methyloxy)phenyl]-2-({[1-(2-fluorophenyl)-6-methyl-1H-pyrazolo[3,4-d]pyrimidin-4-yl]amino}methyl)-4-methyl-2-pentanol;
1,1,1-trifluoro-4-[5-fluoro-2-(methyloxy)phenyl]-2-({[1-(4-fluorophenyl)-6-methyl-1H-pyrazolo[3,4-d]pyrimidin-4-yl]amino}methyl)-4-methyl-2-pentanol;
2-({[1-(2,4-difluorophenyl)-6-methyl-1H-pyrazolo[3,4-d]pyrimidin-4-yl]amino}methyl)-1,1,1-trifluoro-4-[5-fluoro-2-(methyloxy)phenyl]-4-methyl-2-pentanol;
1,1,1-trifluoro-4-[5-fluoro-2-(methyloxy)phenyl]-4-methyl-2-({[6-methyl-1-(3-pyridinyl)-1H-pyrazolo[3,4-d]pyrimidin-4-yl]amino}methyl)-2-pentanol;
1,1,1-trifluoro-4-[5-fluoro-2-(methyloxy)phenyl]-4-methyl-2-({[6-methyl-1-(4-pyridinyl)-1H-pyrazolo[3,4-d]pyrimidin-4-yl]amino}methyl)-2-pentanol;
1,1,1-trifluoro-4-[5-fluoro-2-(methyloxy)phenyl]-4-methyl-2-({[6-methyl-1-(3-thienyl)-1H-pyrazolo[3,4-d]pyrimidin-4-yl]amino}methyl)-2-pentanol;
1,1,1-trifluoro-4-[5-fluoro-2-(methyloxy)phenyl]-2-({[1-(6-fluoro-3-pyridinyl)-6-methyl-1H-pyrazolo[3,4-d]pyrimidin-4-yl]amino}methyl)-4-methyl-2-pentanol;
4-(4-{[4-[5-fluoro-2-(methyloxy)phenyl]-2-hydroxy-4-methyl-2-(trifluoromethyl)pentyl]amino}-6-methyl-1H-pyrazolo[3,4-d]pyrimidin-1-yl)benzonitrile;
ethyl 3-(4-{[4-[5-fluoro-2-(methyloxy)phenyl]-2-hydroxy-4-methyl-2-(trifluoromethyl)pentyl]amino}-6-methyl-1H-pyrazolo[3,4-d]pyrimidin-1-yl)benzoate;
methyl 3-(4-{[4-[5-fluoro-2-(methyloxy)phenyl]-2-hydroxy-4-methyl-2-(trifluoromethyl)pentyl]amino}-6-methyl-1H-pyrazolo[3,4-d]pyrimidin-1-yl)benzoate;
2-({[6-ethyl-1-(4-fluorophenyl)-1H-pyrazolo[3,4-d]pyrimidin-4-yl]amino}methyl)-1,1,1-trifluoro-4-[5-fluoro-2-(methyloxy)phenyl]-4-methyl-2-pentanol;
2-[3-({[1-(2,4-difluorophenyl)-6-methyl-1H-pyrazolo[3,4-d]pyrimidin-4-yl]amino}methyl)-4,4,4-trifluoro-3-hydroxy-1,1-dimethylbutyl]-4-fluorophenol;
2-({[6-ethyl-1-phenyl-1H-pyrazolo[3,4-d]pyrimidin-4-yl]amino}methyl)-1,1,1-trifluoro-4-[5-fluoro-2-(methyloxy)phenyl]-4-methyl-2-pentanol;
3-(4-{[4-[5-fluoro-2-(methyloxy)phenyl]-2-hydroxy-4-methyl-2-(trifluoromethyl)pentyl]amino}-6-methyl-1H-pyrazolo[3,4-d]pyrimidin-1-yl)benzonitrile;
1,1,1-trifluoro-4-[5-fluoro-2-(methyloxy)phenyl]-2-({[1-(2-fluoro-3-pyridinyl)-6-methyl-1H-pyrazolo[3,4-d]pyrimidin-4-yl]amino}methyl)-4-methyl-2-pentanol;
4-(2,3-dihydro-1-benzofuran-7-yl)-2-({[6-ethyl-1-(3-cyanophenyl)-1H-pyrazolo[3,4-d]pyrimidin-4-yl]amino}methyl)-1,1,1-trifluoro-4-methyl-2-pentanol;
methyl 3-(4-{[4-[2,3-dihydro-1-benzofuran-7-yl]-2-hydroxy-4-methyl-2-(trifluoromethyl)pentyl]amino}-6-ethyl-1H-pyrazolo[3,4-d]pyrimidin-1-yl)benzoate; or
   a physiologically functional derivative thereof.

In another embodiment, the compound of formula (I) is:
4-(2,3-dihydro-1-benzofuran-7-yl)-1,1,1-trifluoro-2-({[1-(2-fluorophenyl)-6-methyl-1H-pyrazolo[3,4-d]pyrimidin-4-yl]amino}methyl)-4-methyl-2-pentanol;
2-({[1-(2,4-difluorophenyl)-6-methyl-1H-pyrazolo[3,4-d]pyrimidin-4-yl]amino}methyl)-4-(2,3-dihydro-1-benzofuran-7-yl)-1,1,1-trifluoro-4-methyl-2-pentanol;
2-({[1-(2,4-difluorophenyl)-6-ethyl-1H-pyrazolo[3,4-d]pyrimidin-4-yl]amino}methyl)-4-(2,3-dihydro-1-benzofuran-7-yl)-1,1,1-trifluoro-4-methyl-2-pentanol;
1,1,1-trifluoro-4-[5-fluoro-2-(methyloxy)phenyl]-2-({[1-(2-fluorophenyl)-6-methyl-1H-pyrazolo[3,4-d]pyrimidin-4-yl]amino}methyl)-4-methyl-2-pentanol (Enantiomer 2);
2-({[1-(2,4-difluorophenyl)-6-methyl-1H-pyrazolo[3,4-d]pyrimidin-4-yl]amino}methyl)-1,1,1-trifluoro-4-[5-fluoro-2-(methyloxy)phenyl]-4-methyl-2-pentanol (Enantiomer 1);
1,1,1-trifluoro-4-[5-fluoro-2-(methyloxy)phenyl]-4-methyl-2-({[6-methyl-1-(4-pyridinyl)-1H-pyrazolo[3,4-d]pyrimidin-4-yl]amino}methyl)-2-pentanol;
2-[3-({[1-(2,4-difluorophenyl)-6-methyl-1H-pyrazolo[3,4-d]pyrimidin-4-yl]amino}methyl)-4,4,4-trifluoro-3-hydroxy-1,1-dimethylbutyl]-4-fluorophenol; or
   a physiologically functional derivative thereof.

In a further embodiment, the compound of formula (I) is:
1,1,1-trifluoro-4-[5-fluoro-2-(methyloxy)phenyl]-2-({[1-(2-fluorophenyl)-6-methyl-1H-pyrazolo[3,4-d]pyrimidin-4-yl]amino}methyl)-4-methyl-2-pentanol (Enantiomer 2);
2-({[1-(2,4-difluorophenyl)-6-methyl-1H-pyrazolo[3,4-d]pyrimidin-4-yl]amino}methyl)-1,1,1-trifluoro-4-[5-fluoro-2-(methyloxy)phenyl]-4-methyl-2-pentanol (Enantiomer 1); or
   a physiologically functional derivative thereof.

It will be appreciated by those skilled in the art that at least one isomer (e.g. one enantiomer of the racemate) has the described activity. The other isomers may have similar activity, less activity, no activity or may have some antagonist activity in a functional assay.

The invention includes physiologically functional derivatives of the compound of formula (I). By the term “physiologically functional derivative” is meant a chemical derivative of a compound of formula (I) having the same physiological function as a free compound of formula (I), for example, by being convertible in the body thereto and includes any pharmaceutically acceptable esters, carbonates, carbamates, salts and solvates of compounds of formula (I), and solvates of any pharmaceutically acceptable esters, carbonates, carbamates or salts of compounds of formula (I), which, upon administration to the recipient, are capable of providing (directly or indirectly) compounds of formula (I) or active metabolite or residue thereof. Thus one embodiment of the invention embraces compounds of formula (I) and salts and solvates thereof. Another embodiment of the invention embraces compounds of formula (I) and salts thereof. A further embodiment of the invention embraces compounds of formula (I).

Salts and solvates of the compounds of formula (I) and physiologically functional derivatives thereof which are suitable for use in medicine are those wherein the counter-ion or associated solvent is pharmaceutically acceptable. However, salts and solvates having non-pharmaceutically acceptable counter-ions or associated solvents are within the scope of the present invention, for example, for use as intermediates in the preparation of other compounds of formula (I) and their pharmaceutically acceptable salts, solvates, and physiologically functional derivatives.

Suitable salts according to the invention include those formed with both organic and inorganic acids or bases. Pharmaceutically acceptable acid addition salts include those formed from hydrochloric, hydrobromic, sulphuric, citric, tartaric, phosphoric, lactic, pyruvic, acetic, trifluoroacetic, triphenylacetic, sulphamic, sulphanilic, succinic, oxalic, fumaric, maleic, malic, glutamic, aspartic, oxaloacetic, methanesulphonic, ethanesulphonic, arylsulphonic (for example p-toluenesulphonic, benzenesulphonic, naphthalenesulphonic or naphthalenedisulphonic), salicylic, glutaric, gluconic, tricarballylic, cinnamic, substituted cinnamic (for example, phenyl, methyl, methoxy or halo substituted cinnamic, including 4-methyl and 4-methoxycinnamic acid), ascorbic, oleic, naphthoic, hydroxynaphthoic (for example 1- or 3-hydroxy-2-naphthoic), naphthaleneacrylic (for example naphthalene-2-acrylic), benzoic, 4-methoxybenzoic, 2- or 4-hydroxybenzoic, 4-chlorobenzoic, 4-phenylbenzoic, benzeneacrylic (for example 1,4-benzenediacrylic) and isethionic acids. Pharmaceutically acceptable base salts include ammonium salts, alkali metal salts such as those of sodium and potassium, alkaline earth metal salts such as those of calcium and magnesium and salts with organic bases such as dicyclohexylamine and N-methyl-D-glucamine.

Examples of solvates include hydrates.

The compounds of the invention are expected to have potentially beneficial anti-inflammatory or anti-allergic effects, particularly upon topical administration, demonstrated by, for example, their ability to bind to the glucocorticoid receptor and to illicit a response via that receptor. Hence, the compounds of the invention may be of use in the treatment of inflammatory and/or allergic disorders.

Examples of disease states in which the compounds of the invention are expected to have utility include skin diseases such as eczema, psoriasis, allergic dermatitis, neurodermatitis, pruritis and hypersensitivity reactions; inflammatory conditions of the nose, throat or lungs such as asthma (including allergen-induced asthmatic reactions), rhinitis (including hayfever), nasal polyps, chronic obstructive pulmonary disease (COPD), interstitial lung disease, and fibrosis; inflammatory bowel conditions such as ulcerative colitis and Crohn's disease; and auto-immune diseases such as rheumatoid arthritis.

It will be appreciated by those skilled in the art that reference herein to treatment extends to prophylaxis as well as the treatment of established conditions.

As mentioned above, compounds of the invention are expected to be of use in human or veterinary medicine, in particular as anti-inflammatory and/or anti-allergic agents.

There is thus provided as a further aspect of the invention a compound of the invention for use in human or veterinary medicine, particularly in the treatment of patients with inflammatory and/or allergic conditions, such as rheumatoid arthritis, asthma, COPD, allergy and/or rhinitis.

In another aspect of the invention there is provided a compound of the invention for use in the treatment of patients with skin disease such as eczema, psoriasis, allergic dermatitis, neurodermatitis, pruritis and/or hypersensitivity reactions.

According to another aspect of the invention, there is provided the use of a compound of the invention for the manufacture of a medicament for the treatment of patients with inflammatory and/or allergic conditions, such as rheumatoid arthritis, asthma, COPD, allergy and/or rhinitis.

According to yet to another aspect of the invention, there is provided the use of a compound of the invention for the manufacture of a medicament for the treatment of patients with skin disease such as eczema, psoriasis, allergic dermatitis, neurodermatitis, pruritis and/or hypersensitivity reactions.

In a further or alternative aspect, there is provided a method for the treatment of a human or animal subject with an inflammatory and/or allergic condition such as rheumatoid arthritis, asthma, COPD, allergy and/or rhinitis, which method comprises administering to said human or animal subject an effective amount of a compound of the invention.

In yet a further or alternative aspect, there is provided a method for the treatment of a human or animal subject with skin disease such as eczema, psoriasis, allergic dermatitis, neurodermatitis, pruritis and/or hypersensitivity reactions, which method comprises administering to said human or animal subject an effective amount of a compound of the invention.

The compounds of the invention may be formulated for administration in any convenient way, and the invention therefore also includes within its scope pharmaceutical compositions comprising a compound of the invention together, if desirable, in admixture with one or more physiologically acceptable diluents or carriers.

Further, there is provided a process for the preparation of such pharmaceutical compositions which comprises mixing the ingredients.

The compounds of the invention may, for example, be formulated for oral, buccal, sublingual, parenteral, local rectal administration or other local administration.

Local administration as used herein, includes administration by insufflation and inhalation. Examples of various types of preparation for local administration include ointments, lotions, creams, gels, foams, preparations for delivery by transdermal patches, powders, sprays, aerosols, capsules or cartridges for use in an inhaler or insufflator or drops (eg. eye or nose drops), solutions/suspensions for nebulisation, suppositories, pessaries, retention enemas and chewable or suckable tablets or pellets (eg. for the treatment of aphthous ulcers) or liposome or microencapsulation preparations.

Formulations for administration topically to the nose for example, for the treatment of rhinitis, include pressurised aerosol formulations and aqueous formulations administered to the nose by pressurised pump. Formulations which are non-pressurised and adapted to be administered topically to the nasal cavity are of particular interest. Suitable formulations contain water as the diluent or carrier for this purpose. Aqueous formulations for administration to the lung or nose may be provided with conventional excipients such as buffering agents, tonicity modifying agents and the like. Aqueous formulations may also be administered to the nose by nebulisation.

The compounds of the invention may be formulated as a fluid formulation for delivery from a fluid dispenser, for example a fluid dispenser having a dispensing nozzle or dispensing orifice through which a metered dose of the fluid formulation is dispensed upon the application of a user-applied force to a pump mechanism of the fluid dispenser. Such fluid dispensers are generally provided with a reservoir of multiple metered doses of the fluid formulation, the doses being dispensable upon sequential pump actuations. The dispensing nozzle or orifice may be configured for insertion into the nostrils of the user for spray dispensing of the fluid formulation into the nasal cavity. A fluid dispenser of the aforementioned type is described and illustrated in WO05/044354, the entire content of which is hereby incorporated herein by reference. The dispenser has a housing which houses a fluid discharge device having a compression pump mounted on a container for containing a fluid formulation. The housing has at least one finger-operable side lever which is movable inwardly with respect to the housing to cam the container upwardly in the housing to cause the pump to compress and pump a metered dose of the formulation out of a pump stem through a nasal nozzle of the housing. In one embodiment, the fluid dispenser is of the general type illustrated in FIGS. 30-40 of WO05/044354.

Ointments, creams and gels, may, for example, be formulated with an aqueous or oily base with the addition of suitable thickening and/or gelling agent and/or solvents. Such bases may thus, for example, include water and/or an oil such as liquid paraffin or a vegetable oil such as arachis oil or castor oil, or a solvent such as polyethylene glycol. Thickening agents and gelling agents which may be used according to the nature of the base include soft paraffin, aluminium stearate, cetostearyl alcohol, polyethylene glycols, woolfat, beeswax, carboxypolymethylene and cellulose derivatives, and/or glyceryl monostearate and/or non-ionic emulsifying agents.

Lotions may be formulated with an aqueous or oily base and will in general also contain one or more emulsifying agents, stabilising agents, dispersing agents, suspending agents or thickening agents.

Powders for external application may be formed with the aid of any suitable powder base, for example, talc, lactose or starch. Drops may be formulated with an aqueous or non-aqueous base also comprising one or more dispersing agents, solubilising agents, suspending agents or preservatives.

Spray compositions may for example be formulated as aqueous solutions or suspensions or as aerosols delivered from pressurised packs, such as a metered dose inhaler, with the use of a suitable liquefied propellant. Aerosol compositions suitable for inhalation can be either a suspension or a solution and generally contain a compound of formula (I) and a suitable propellant such as a fluorocarbon or hydrogen-containing chlorofluorocarbon or mixtures thereof, particularly hydrofluoroalkanes, especially 1,1,1,2-tetrafluoroethane, 1,1,1,2,3,3,3-heptafluoro-n-propane or a mixture thereof. The aerosol composition may optionally contain additional formulation excipients well known in the art such as surfactants eg. oleic acid, lecithin or an oligolactic acid or derivative eg. as described in WO94/21229 and WO98/34596, and cosolvents eg. ethanol.

There is thus provided as a further aspect of the invention a pharmaceutical aerosol formulation comprising a compound of the invention and a fluorocarbon or hydrogen-containing chlorofluorocarbon as propellant, optionally in combination with a surfactant and/or a cosolvent.

According to another aspect of the invention, there is provided a pharmaceutical aerosol formulation wherein the propellant is selected from 1,1,1,2-tetrafluoroethane, 1,1,1,2,3,3,3-heptafluoro-n-propane and mixtures thereof.

The formulations of the invention may be buffered by the addition of suitable buffering agents.

Capsules and cartridges for use in an inhaler or insufflator, of for example gelatine, may be formulated containing a powder mix for inhalation of a compound of the invention and a suitable powder base such as lactose or starch. Each capsule or cartridge may generally contain from 20 μg to 10 mg of the compound of formula (I). Alternatively, the compound of the invention may be presented without excipients such as lactose.

The proportion of the active compound of formula (I) in the local compositions according to the invention depends on the precise type of formulation to be prepared but will generally be within the range of from 0.001 to 10% by weight. Generally, for most types of preparations, the proportion used will normally be within the range of from 0.005 to 1%, for example from 0.01 to 0.5%. However, in powders for inhalation or insufflation the proportion used will normally be within the range of from 0.1 to 5%.

Aerosol formulations are preferably arranged so that each metered dose or “puff” of aerosol contains from 20 μg to 10 mg, preferably from 20 μg to 2000 μg, more preferably from about 20 μg to 500 μg of a compound of formula (I). Administration may be once daily or several times daily, for example 2, 3, 4 or 8 times, giving for example 1, 2 or 3 doses each time. The overall daily dose with an aerosol will be within the range from 100 μg to 10 mg, preferably from 200 μg to 2000 μg. The overall daily dose and the metered dose delivered by capsules and cartridges in an inhaler or insufflator will generally be double that delivered with aerosol formulations.

In the case of suspension aerosol formulations, the particle size of the particular (e.g., micronised) drug should be such as to permit inhalation of substantially all the drug into the lungs upon administration of the aerosol formulation and will thus be less than 100 microns, desirably less than 20 microns, and, in particular, in the range of from 1 to 10 microns, such as from 1 to 5 microns, more preferably from 2 to 3 microns.

The formulations of the invention may be prepared by dispersal or dissolution of the medicament and a compound of the invention in the selected propellant in an appropriate container, for example, with the aid of sonication or a high-shear mixer. The process is desirably carried out under controlled humidity conditions.

The chemical and physical stability and the pharmaceutical acceptability of the aerosol formulations according to the invention may be determined by techniques well known to those skilled in the art. Thus, for example, the chemical stability of the components may be determined by HPLC assay, for example, after prolonged storage of the product. Physical stability data may be gained from other conventional analytical techniques such as, for example, by leak testing, by valve delivery assay (average shot weights per actuation), by dose reproducibility assay (active ingredient per actuation) and spray distribution analysis.

The stability of the suspension aerosol formulations according to the invention may be measured by conventional techniques, for example, by measuring flocculation size distribution using a back light scattering instrument or by measuring particle size distribution by cascade impaction or by the “twin impinger” analytical process. As used herein reference to the “twin impinger” assay means “Determination of the deposition of the emitted dose in pressurised inhalations using apparatus A” as defined in British Pharmacopaeia 1988, pages A204-207, Appendix XVII C. Such techniques enable the “respirable fraction” of the aerosol formulations to be calculated. One method used to calculate the “respirable fraction” is by reference to “fine particle fraction” which is the amount of active ingredient collected in the lower impingement chamber per actuation expressed as a percentage of the total amount of active ingredient delivered per actuation using the twin impinger method described above.

The term “metered dose inhaler” or MDI means a unit comprising a can, a secured cap covering the can and a formulation metering valve situated in the cap. MDI system includes a suitable channelling device. Suitable channelling devices comprise for example, a valve actuator and a cylindrical or cone-like passage through which medicament may be delivered from the filled canister via the metering valve to the nose or mouth of a patient such as a mouthpiece actuator.

MDI canisters generally comprise a container capable of withstanding the vapour pressure of the propellant used such as a plastic or plastic-coated glass bottle or preferably a metal can, for example, aluminium or an alloy thereof which may optionally be anodised, lacquer-coated and/or plastic-coated (for example incorporated herein by reference WO96/32099 wherein part or all of the internal surfaces are coated with one or more fluorocarbon polymers optionally in combination with one or more non-fluorocarbon polymers), which container is closed with a metering valve. The cap may be secured onto the can via ultrasonic welding, screw fitting or crimping. MDIs taught herein may be prepared by methods of the art (eg. see Byron, above and WO96/32099). Preferably the canister is fitted with a cap assembly, wherein a drug-metering valve is situated in the cap, and said cap is crimped in place.

In one embodiment of the invention the metallic internal surface of the can is coated with a fluoropolymer, most preferably blended with a non-fluoropolymer. In another embodiment of the invention the metallic internal surface of the can is coated with a polymer blend of polytetrafluoroethylene (PTFE) and polyethersulfone (PES). In a further embodiment of the invention the whole of the metallic internal surface of the can is coated with a polymer blend of polytetrafluoroethylene (PTFE) and polyethersulfone (PES).

The metering valves are designed to deliver a metered amount of the formulation per actuation and incorporate a gasket to prevent leakage of propellant through the valve. The gasket may comprise any suitable elastomeric material such as, for example, low density polyethylene, chlorobutyl, bromobutyl, EPDM, black and white butadiene-acrylonitrile rubbers, butyl rubber and neoprene. Suitable valves are commercially available from manufacturers well known in the aerosol industry, for example, from Valois, France (eg. DF10, DF30, DF60), Bespak plc, UK (eg. BK300, BK357) and 3M-Neotechnic Ltd, UK (eg. Spraymiser™).

In various embodiments, the MDIs may also be used in conjunction with other structures such as, without limitation, overwrap packages for storing and containing the MDIs, including those described in U.S. Pat. Nos. 6,119,853; 6,179,118; 6,315,112; 6,352,152; 6,390,291; and 6,679,374, as well as dose counter units such as, but not limited to, those described in U.S. Pat. Nos. 6,360,739 and 6,431,168.

Conventional bulk manufacturing methods and machinery well known to those skilled in the art of pharmaceutical aerosol manufacture may be employed for the preparation of large-scale batches for the commercial production of filled canisters. Thus, for example, in one bulk manufacturing method for preparing suspension aerosol formulations a metering valve is crimped onto an aluminium can to form an empty canister. The particulate medicament is added to a charge vessel and liquefied propellant together with the optional excipients is pressure filled through the charge vessel into a manufacturing vessel. The drug suspension is mixed before recirculation to a filling machine and an aliquot of the drug suspension is then filled through the metering valve into the canister. In one example bulk manufacturing method for preparing solution aerosol formulations a metering valve is crimped onto an aluminium can to form an empty canister. The liquefied propellant together with the optional excipients and the dissolved medicament is pressure filled through the charge vessel into a manufacturing vessel.

In an alternative process, an aliquot of the liquefied formulation is added to an open canister under conditions which are sufficiently cold to ensure the formulation does not vaporise, and then a metering valve crimped onto the canister.

Typically, in batches prepared for pharmaceutical use, each filled canister is check-weighed, coded with a batch number and packed into a tray for storage before release testing.

Topical preparations may be administered by one or more applications per day to the affected area; over skin areas occlusive dressings may advantageously be used. Continuous or prolonged delivery may be achieved by an adhesive reservoir system.

For internal administration the compounds according to the invention may, for example, be formulated in conventional manner for oral, nasal, parenteral or rectal administration. Formulations for oral administration include syrups, elixirs, powders, granules, tablets and capsules which typically contain conventional excipients such as binding agents, fillers, lubricants, disintegrants, wetting agents, suspending agents, emulsifying agents, preservatives, buffer salts, flavouring, colouring and/or sweetening agents as appropriate. Dosage unit forms may be preferred as described below.

The compounds of the invention may in general be given by internal administration in cases wherein systemic glucocorticoid receptor agonist therapy is indicated.

Slow release or enteric coated formulations may be advantageous, particularly for the treatment of inflammatory bowel disorders.

In some embodiments, the compounds of the invention will be formulated for oral administration. In other embodiments, the compounds of the invention will be formulated for inhaled administration.

The compounds and pharmaceutical formulations according to the invention may be used in combination with or include one or more other therapeutic agents, for example selected from anti-inflammatory agents, anticholinergic agents (particularly an M₁/M₂/M₃ receptor antagonist), β₂-adrenoreceptor agonists, antiinfective agents such as antibiotics or antivirals, or antihistamines. The invention thus provides, in a further aspect, a combination comprising a compound of the invention together with one or more other therapeutically active agents, for example selected from an anti-inflammatory agent such as a corticosteroid or an NSAID, an anticholinergic agent, a β₂-adrenoreceptor agonist, an antiinfective agent such as an antibiotic or an antiviral, or an antihistamine. One embodiment of the invention encompasses combinations comprising a compound of the invention together with a β₂-adrenoreceptor agonist, and/or an anticholinergic, and/or a PDE-4 inhibitor, and/or an antihistamine.

One embodiment of the invention encompasses combinations comprising one or two other therapeutic agents.

It will be clear to a person skilled in the art that, where appropriate, the other therapeutic ingredient(s) may be used in the form of salts, for example as alkali metal or amine salts or as acid addition salts, or prodrugs, or as esters, for example lower alkyl esters, or as solvates, for example hydrates, to optimise the activity and/or stability and/or physical characteristics, such as solubility, of the therapeutic ingredient. It will be clear also that, where appropriate, the therapeutic ingredients may be used in optically pure form.

In one embodiment, the invention encompasses a combination comprising a compound of the invention together with a β₂-adrenoreceptor agonist.

Examples of β₂-adrenoreceptor agonists include salmeterol (which may be a racemate or a single enantiomer, such as the R-enantiomer), salbutamol (which may be a racemate or a single enantiomer such as the R-enantiomer), formoterol (which may be a racemate or a single diastereomer such as the R,R-diastereomer), salmefamol, fenoterol, carmoterol, etanterol, naminterol, clenbuterol, pirbuterol, flerobuterol, reproterol, bambuterol, indacaterol, terbutaline, 3-(4-{[6-({(2R)-2-hydroxy-2-[4-hydroxy-3-(hydroxymethyl)phenyl]ethyl}amino)hexyl]oxy}butyl)benzenesulfonamide, 3-(3-{[7-({(2R)-2-hydroxy-2-[4-hydroxy-3-hydroxymethyl)phenyl]ethyl}-amino)heptyl]oxy}propyl)benzenesulfonamide, 4-{(1R)-2-[(6-{2-[(2,6-dichlorobenzyl)oxy]ethoxy} hexyl)amino]-1-hydroxyethyl}-2-(hydroxymethyl)phenol, 4-{(1R)-2-[(6-{4-[3-(cyclopentylsulfonyl)phenyl]butoxy}hexyl)amino]-1-hydroxyethyl}-2-(hydroxymethyl)phenol, N-[2-hydroxyl-5-[(1R)-1-hydroxy-2-[[2-4-[[(2R)-2-hydroxy-2-phenylethyl]amino]phenyl]ethyl]amino]ethyl]phenyl]formamide, N-2{2-[4-(3-phenyl-4-methoxyphenyl)aminophenyl]ethyl}-2-hydroxy-2-(8-hydroxy-2(1H)-quinolinon-5-yl)ethylamine, 5-[(R)-2-(2-{4-[4-(2-amino-2-methyl-propoxy)-phenylamino]-phenyl}-ethylamino)-1-hydroxy-ethyl]-8-hydroxy-1H-quinolin-2-one and salts thereof, for example the xinafoate salt of salmeterol, the sulphate salt or free base of salbutamol or the fumarate salt of formoterol. In one embodiment, the β2-adrenoreceptor agonists are long-acting β₂-adrenoreceptor agonists, for example those having a therapeutic effect over a 24 hour period.

Examples of β₂-adrenoreceptor agonists may include those described in WO02/66422A, WO02/070490, WO02/076933, WO03/024439, WO03/072539, WO 03/091204, WO04/016578, WO04/022547, WO04/037807, WO04/037773, WO04/037768, WO04/039762, WO04/039766, WO01/42193 and WO03/042160.

The β₂-adrenoreceptor agonist may be in the form of a salt formed with a pharmaceutically acceptable acid selected from sulphuric, hydrochloric, fumaric, hydroxynaphthoic (for example 1- or 3-hydroxy-2-naphthoic), cinnamic, substituted cinnamic, triphenylacetic, sulphamic, sulphanilic, naphthaleneacrylic, benzoic, 4-methoxybenzoic, 2- or 4-hydroxybenzoic, 4-chlorobenzoic and 4-phenylbenzoic acid.

Suitable anti-inflammatory agents include corticosteroids. Examples of corticosteroids which may be used in combination with the compounds of the invention are those oral and inhaled corticosteroids and their pro-drugs which have anti-inflammatory activity. Examples include methyl prednisolone, prednisolone, dexamethasone, fluticasone propionate, 6α,9α-difluoro-11β-hydroxy-16α-methyl-17α-[(4-methyl-1,3-thiazole-5-carbonyl)oxy]-3-oxo-androsta-1,4-diene-17β-carbothioic acid S-fluoromethyl ester, 6α,9α-difluoro-17α-[(2-furanylcarbonyl)oxy]-11β-hydroxy-16α-methyl-3-oxo-androsta-1,4-diene-17β-carbothioic acid S-fluoromethyl ester (fluticasone furoate), 6α,9α-difluoro-11β-hydroxy-16α-methyl-3-oxo-17α-propionyloxy-androsta-1,4-diene-17β-carbothioic acid S-(2-oxo-tetrahydro-furan-3S-yl)ester, 6α,9α-difluoro-11β-hydroxy-16α-methyl-3-oxo-17α-(2,2,3,3-tetramethycyclopropylcarbonyl)oxy-androsta-1,4-diene-17β-carbothioic acid S-cyanomethyl ester and 6α,9α-difluoro-11β-hydroxy-16α-methyl-17α-(1-methycyclopropylcarbonyl)oxy-3-oxo-androsta-1,4-diene-17β-carbothioic acid S-fluoromethyl ester, beclomethasone esters (for example the 17-propionate ester or the 17,21-dipropionate ester), budesonide, flunisolide, mometasone esters (for example mometasone furoate), triamcinolone acetonide, rofleponide, ciclesonide (16α,17-[[(R)-cyclohexylmethylene]bis(oxy)]-11β,21-dihydroxy-pregna-1,4-diene-3,20-dione), butixocort propionate, RPR-106541, and ST-126. In one embodiment corticosteroids include fluticasone propionate, 6α,9α-difluoro-11β-hydroxy-16α-methyl-17α-[(4-methyl-1,3-thiazole-5-carbonyl)oxy]-3-oxo-androsta-1,4-diene-17β-carbothioic acid S-fluoromethyl ester, 6α,9α-difluoro-17α-[(2-furanylcarbonyl)oxy]-11β-hydroxy-16α-methyl-3-oxo-androsta-1,4-diene-17β-carbothioic acid S-fluoromethyl ester, 6α,9α-difluoro-11β-hydroxy-16α-methyl-3-oxo-17α-(2,2,3,3-tetramethycyclopropylcarbonyl)oxy-androsta-1,4-diene-17β-carbothioic acid S-cyanomethyl ester and 6α,9α-difluoro-11β-hydroxy-16α-methyl-17α-(1-methycyclopropylcarbonyl)oxy-3-oxo-androsta-1,4-diene-17β-carbothioic acid S-fluoromethyl ester. In one embodiment the corticosteroid is 6α,9α-difluoro-17α-[(2-furanylcarbonyl)oxy]-11β-hydroxy-16α-methyl-3-oxo-androsta-1,4-diene-17β-carbothioic acid S-fluoromethyl ester.

Examples of corticosteroids may include those described in WO02/088167, WO02/100879, WO02/12265, WO02/12266, WO05/005451, WO05/005452, WO06/072599 and WO06/072600.

Non-steroidal compounds having glucocorticoid agonism that may possess selectivity for transrepression over transactivation and that may be useful in combination therapy include those covered in the following published patent applications and patents: WO03/082827, WO98/54159, WO04/005229, WO04/009017, WO04/018429, WO03/104195, WO03/082787, WO03/082280, WO03/059899, WO03/101932, WO02/02565, WO01/16128, WO00/66590, WO03/086294, WO04/026248, WO03/061651, WO03/08277, WO06/000401, WO06/000398 and WO06/015870.

Examples of anti-inflammatory agents include non-steroidal anti-inflammatory drugs (NSAID's).

Examples of NSAID's include sodium cromoglycate, nedocromil sodium, phosphodiesterase (PDE) inhibitors (for example, theophylline, PDE4 inhibitors or mixed PDE3/PDE4 inhibitors), leukotriene antagonists, inhibitors of leukotriene synthesis (for example, montelukast), iNOS inhibitors, tryptase and elastase inhibitors, beta-2 integrin antagonists and adenosine receptor agonists or antagonists (for example, adenosine 2a agonists), cytokine antagonists (for example, chemokine antagonists, such as a CCR3 antagonist) or inhibitors of cytokine synthesis, or 5-lipoxygenase inhibitors. An iNOS (inducible nitric oxide synthase inhibitor) is preferably for oral administration. Examples of iNOS inhibitors include those disclosed in WO93/13055, WO98/30537, WO02/50021, WO95/34534 and WO99/62875. Examples of CCR3 inhibitors include those disclosed in WO02/26722.

In one embodiment, the invention provides the use of the compounds of the invention in combination with a phosphodiesterase 4 (PDE4) inhibitor, for example in the case of a formulation adapted for inhalation. The PDE4-specific inhibitor may be any compound that is known to inhibit the PDE4 enzyme or which is discovered to act as a PDE4 inhibitor, and which are only PDE4 inhibitors, not compounds which inhibit other members of the PDE family, such as PDE3 and PDE5, as well as PDE4.

Compounds include cis-4-cyano-4-(3-cyclopentyloxy-4-methoxyphenyl)cyclohexan-1-carboxylic acid, 2-carbomethoxy-4-cyano-4-(3-cyclopropylmethoxy-4-difluoromethoxyphenyl)cyclohexan-1-one and cis-[4-cyano-4-(3-cyclopropylmethoxy-4-difluoromethoxyphenyl)cyclohexan-1-ol]. Another compound is cis-4-cyano-4-[3-(cyclopentyloxy)-4-methoxyphenyl]cyclohexane-1-carboxylic acid (also known as cilomilast) and its salts, esters, pro-drugs or physical forms, which is described in U.S. Pat. No. 5,552,438 issued 3 Sep. 1996; this patent and the compounds it discloses are incorporated herein in full by reference.

Other compounds include AWD-12-281 from Elbion (Hofgen, N. et al. 15th EFMC Int Symp Med Chem (September 6-10, Edinburgh) 1998, Abst P. 98; CAS reference No. 247584020-9); a 9-benzyladenine derivative nominated NCS-613 (INSERM); D-4418 from Chiroscience and Schering-Plough; a benzodiazepine PDE4 inhibitor identified as CI-1018 (PD-168787) and attributed to Pfizer; a benzodioxole derivative disclosed by Kyowa Hakko in WO99/16766; K-34 from Kyowa Hakko; V-11294A from Napp (Landells, L. J. et al. Eur Resp J [Annu Cong Eur Resp Soc (September 19-23, Geneva) 1998] 1998, 12 (Suppl. 28): Abst P2393); roflumilast (CAS reference No 162401-32-3) and a pthalazinone (WO99/47505, the disclosure of which is hereby incorporated by reference) from Byk-Gulden; Pumafentrine, (−)-p-[(4aR*,10bS*)-9-ethoxy-1,2,3,4,4a,10b-hexahydro-8-methoxy-2-methylbenzo[c][1,6]naphthyridin-6-yl]-N,N-diisopropylbenzamide which is a mixed PDE3/PDE4 inhibitor which has been prepared and published on by Byk-Gulden, now Altana; arofylline under development by Almirall-Prodesfarma; VM554/UM565 from Vernalis; or T-440 (Tanabe Seiyaku; Fuji, K. et al. J Pharmacol Exp Ther, 1998, 284(1): 162), and T2585.

Further compounds are disclosed in the published international patent application WO04/024728 (Glaxo Group Ltd), WO04/056823 (Glaxo Group Ltd) and WO04/103998 (Glaxo Group Ltd).

Examples of anticholinergic agents are those compounds that act as antagonists at the muscarinic receptors, in particular those compounds which are antagonists of the M₁ or M₃ receptors, dual antagonists of the M₁/M₃ or M₂/M₃, receptors or pan-antagonists of the M₁/M₂/M₃ receptors. Exemplary compounds for administration via inhalation include ipratropium (for example, as the bromide, CAS 22254-24-6, sold under the name Atrovent), oxitropium (for example, as the bromide, CAS 30286-75-0) and tiotropium (for example, as the bromide, CAS 136310-93-5, sold under the name Spiriva). Also of interest are revatropate (for example, as the hydrobromide, CAS 262586-79-8) and LAS-34273 which is disclosed in WO01/04118. Exemplary compounds for oral administration include pirenzepine (for example, CAS 28797-61-7), darifenacin (for example, CAS 133099-04-4, or CAS 133099-07-7 for the hydrobromide sold under the name Enablex), oxybutynin (for example, CAS 5633-20-5, sold under the name Ditropan), terodiline (for example, CAS 15793-40-5), tolterodine (for example, CAS 124937-51-5, or CAS 124937-52-6 for the tartrate, sold under the name Detrol), otilonium (for example, as the bromide, CAS 26095-59-0, sold under the name Spasmomen), trospium chloride (for example, CAS 10405-02-4) and solifenacin (for example, CAS 242478-37-1, or CAS 242478-38-2, or the succinate also known as YM-905 and sold under the name Vesicare).

Other anticholinergic agents include compounds of formula (XXI), which are disclosed in U.S. patent application 60/487,981:

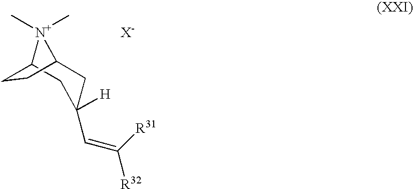

in which the preferred orientation of the alkyl chain attached to the tropane ring is endo; R³¹ and R³² are, independently, selected from the group consisting of straight or branched chain lower alkyl groups having preferably from 1 to 6 carbon atoms, cycloalkyl groups having from 5 to 6 carbon atoms, cycloalkyl-alkyl having from 6 to 10 carbon atoms, 2-thienyl, 2-pyridyl, phenyl, phenyl substituted with an alkyl group having not in excess of 4 carbon atoms and phenyl substituted with an alkoxy group having not in excess of 4 carbon atoms;
X⁻ represents an anion associated with the positive charge of the N atom. X⁻ may be but is not limited to chloride, bromide, iodide, sulfate, benzene sulfonate, and toluene sulfonate, including, for example:
(3-endo)-3-(2,2-di-2-thienylethenyl)-8,8-dimethyl-8-azoniabicyclo[3.2.1]octane bromide;
(3-endo)-3-(2,2-diphenylethenyl)-8,8-dimethyl-8-azoniabicyclo[3.2.1]octane bromide;
(3-endo)-3-(2,2-diphenylethenyl)-8,8-dimethyl-8-azoniabicyclo[3.2.1]octane 4-methylbenzenesulfonate;
(3-endo)-8,8-dimethyl-3-[2-phenyl-2-(2-thienyl)ethenyl]-8-azoniabicyclo[3.2.1]octane bromide; and/or
(3-endo)-8,8-dimethyl-3-[2-phenyl-2-(2-pyridinyl)ethenyl]-8-azoniabicyclo[3.2.1]octane bromide.

Further anticholinergic agents include compounds of formula (XXII) or (XXIII), which are disclosed in U.S. patent application 60/511,009:

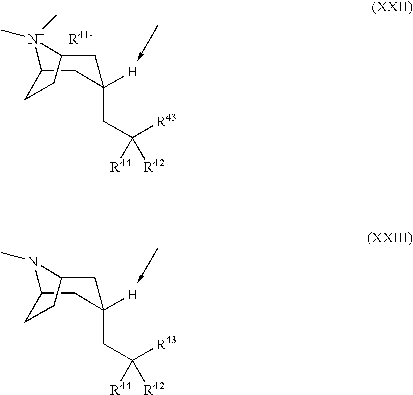

wherein:
the H atom indicated is in the exo position;
R⁴¹⁻ represents an anion associated with the positive charge of the N atom; R⁴¹⁻ may be but is not limited to chloride, bromide, iodide, sulfate, benzene sulfonate and toluene sulfonate;
R⁴² and R⁴³ are independently selected from the group consisting of straight or branched chain lower alkyl groups (having preferably from 1 to 6 carbon atoms), cycloalkyl groups (having from 5 to 6 carbon atoms), cycloalkyl-alkyl (having from 6 to 10 carbon atoms), heterocycloalkyl (having from 5 to 6 carbon atoms) and N or O as the heteroatom, heterocycloalkyl-alkyl (having from 6 to 10 carbon atoms) and N or O as the heteroatom, aryl, optionally substituted aryl, heteroaryl, and optionally substituted heteroaryl;
R⁴⁴ is selected from the group consisting of (C₁-C₆)alkyl, (C₃-C₁₂)cycloalkyl, (C₃-C₇)heterocycloalkyl, (C₁-C₆)alkyl(C₃-C₁₂)cycloalkyl, (C₁-C₆)alkyl(C₃-C₇)heterocycloalkyl, aryl, heteroaryl, (C₁-C₆)alkyl-aryl, (C₁-C₆)alkyl-heteroaryl, —OR⁴⁵, —CH₂OR⁴⁵, —CH₂OH, —CN, —CF₃, —CH₂O(CO)R⁴⁶, —CO₂R⁴⁷, —CH₂NH₂, —CH₂N(R⁴⁷)SO₂R⁴⁵, —SO₂N(R⁴⁷)(R⁴⁸), —CON(R⁴⁷)(R⁴⁸), —CH₂N(R⁴⁸)CO(R⁴⁶), —CH₂N(R⁴⁸)SO₂(R⁴⁶), —CH₂N(R⁴⁸)CO₂(R⁴⁵), —CH₂N(R⁴⁸)CONH(R⁴⁷);
R⁴⁵ is selected from the group consisting of (C₁-C₆)alkyl, (C₁-C₆)alkyl(C₃-C₁₂)cycloalkyl, (C₁-C₆)alkyl(C₃-C₇)heterocycloalkyl, (C₁-C₆)alkyl-aryl, (C₁-C₆)alkyl-heteroaryl;
R⁴⁶ is selected from the group consisting of (C₁-C₆)alkyl, (C₃-C₁₂)cycloalkyl, (C₃-C₇)heterocycloalkyl, (C₁-C₆)alkyl(C₃-C₁₂)cycloalkyl, (C₁-C₆)alkyl(C₃-C₇)heterocycloalkyl, aryl, heteroaryl, (C₁-C₆)alkyl-aryl, (C₁-C₆)alkyl-heteroaryl;
R⁴⁷ and R⁴⁸ are, independently, selected from the group consisting of H, (C₁-C₆)alkyl, (C₃-C₁₂)cycloalkyl, (C₃-C₇)heterocycloalkyl, (C₁-C₆)alkyl(C₃-C₁₂)cycloalkyl, (C₁-C₆)alkyl(C₃-C₇)heterocycloalkyl, (C₁-C₆)alkyl-aryl, and (C₁-C₆)alkyl-heteroaryl, including, for example:
(endo)-3-(2-methoxy-2,2-di-thiophen-2-yl-ethyl)-8,8-dimethyl-8-azonia-bicyclo[3.2.1]octane iodide;
3-((endo)-8-methyl-8-aza-bicyclo[3.2.1]oct-3-yl)-2,2-diphenyl-propionitrile;
(endo)-8-methyl-3-(2,2,2-triphenyl-ethyl)-8-aza-bicyclo[3.2.1]octane;
3-((endo)-8-methyl-8-aza-bicyclo[3.2.1]oct-3-yl)-2,2-diphenyl-propionamide;
3-((endo)-8-methyl-8-aza-bicyclo[3.2.1]oct-3-yl)-2,2-diphenyl-propionic acid;
(endo)-3-(2-cyano-2,2-diphenyl-ethyl)-8,8-dimethyl-8-azonia-bicyclo[3.2.1]octane iodide;
(endo)-3-(2-cyano-2,2-diphenyl-ethyl)-8,8-dimethyl-8-azonia-bicyclo[3.2.1]octane bromide;
3-((endo)-8-methyl-8-aza-bicyclo[3.2.1]oct-3-yl)-2,2-diphenyl-propan-1-ol;
N-benzyl-3-((endo)-8-methyl-8-aza-bicyclo[3.2.1]oct-3-yl)-2,2-diphenyl-propionamide;
(endo)-3-(2-carbamoyl-2,2-diphenyl-ethyl)-8,8-dimethyl-8-azonia-bicyclo[3.2.1]octane iodide;
1-benzyl-3-[3-((endo)-8-methyl-8-aza-bicyclo[3.2.1]oct-3-yl)-2,2-diphenyl-propyl]-urea;
1-ethyl-3-[3-((endo)-8-methyl-8-aza-bicyclo[3.2.1]oct-3-yl)-2,2-diphenyl-propyl]-urea;
N-[3-((endo)-8-methyl-8-aza-bicyclo[3.2.1]oct-3-yl)-2,2-diphenyl-propyl]-acetamide;
N-[3-((endo)-8-methyl-8-aza-bicyclo[3.2.1]oct-3-yl)-2,2-diphenyl-propyl]-benzamide;
3-((endo)-8-methyl-8-aza-bicyclo[3.2.1]oct-3-yl)-2,2-di-thiophen-2-yl-propionitrile;
(endo)-3-(2-cyano-2,2-di-thiophen-2-yl-ethyl)-8,8-dimethyl-8-azonia-bicyclo[3.2.1]octane iodide;
N-[3-((endo)-8-methyl-8-aza-bicyclo[3.2.1]oct-3-yl)-2,2-diphenyl-propyl]-benzenesulfonamide;
[3-((endo)-8-methyl-8-aza-bicyclo[3.2.1]oct-3-yl)-2,2-diphenyl-propyl]-urea;
N-[3-((endo)-8-methyl-8-aza-bicyclo[3.2.1]oct-3-yl)-2,2-diphenyl-propyl]-methanesulfonamide; and/or
(endo)-3-{2,2-diphenyl-3-[(1-phenyl-methanoyl)-amino]-propyl}-8,8-dimethyl-8-azonia-bicyclo[3.2.1]octane bromide.

Further compounds include:
(endo)-3-(2-methoxy-2,2-di-thiophen-2-yl-ethyl)-8,8-dimethyl-8-azonia-bicyclo[3.2.1]octane iodide;
(endo)-3-(2-cyano-2,2-diphenyl-ethyl)-8,8-dimethyl-8-azonia-bicyclo[3.2.1]octane iodide;
(endo)-3-(2-cyano-2,2-diphenyl-ethyl)-8,8-dimethyl-8-azonia-bicyclo[3.2.1]octane bromide;
(endo)-3-(2-carbamoyl-2,2-diphenyl-ethyl)-8,8-dimethyl-8-azonia-bicyclo[3.2.1]octane iodide;
(endo)-3-(2-cyano-2,2-di-thiophen-2-yl-ethyl)-8,8-dimethyl-8-azonia-bicyclo[3.2.1]octane iodide; and/or
(endo)-3-{2,2-diphenyl-3-[(1-phenyl-methanoyl)-amino]-propyl}-8,8-dimethyl-8-azonia-bicyclo[3.2.1]octane bromide.

Examples of antihistamines (also referred to as H1-receptor antagonists) include any one or more of the numerous antagonists known which inhibit H1-receptors, and are safe for human use. First generation antagonists, include derivatives of ethanolamines, ethylenediamines, and alkylamines, such as diphenylhydramine, pyrilamine, clemastine, chlorpheniramine. Second generation antagonists, which are non-sedating, include loratidine, desloratidine, terfenadine, astemizole, acrivastine, azelastine, levocetirizine fexofenadine and cetirizine.

Examples of H1 antagonists include, without limitation, amelexanox, astemizole, azatadine, azelastine, acrivastine, brompheniramine, cetirizine, levocetirizine, efletirizine, chlorpheniramine, clemastine, cyclizine, carebastine, cyproheptadine, carbinoxamine, descarboethoxyloratadine, doxylamine, dimethindene, ebastine, epinastine, efletirizine, fexofenadine, hydroxyzine, ketotifen, loratadine, levocabastine, mizolastine, mequitazine, mianserin, noberastine, meclizine, norastemizole, olopatadine, picumast, pyrilamine, promethazine, terfenadine, tripelennamine, temelastine, trimeprazine and triprolidine, particularly cetirizine, levocetirizine, efletirizine and fexofenadine. In another embodiment, examples of anti-histamines include loratidine, desloratidine, fexofenadine and cetirizine. In a further embodiment the invention provides a combination comprising a compound of formula (I), or a pharmaceutically acceptable salt thereof together with an H3 antagonist (and/or inverse agonist). Examples of H3 antagonists include, for example, those compounds disclosed in WO2004/035556 and in WO2006/045416. Other histamine receptor antagonists which may be used in combination with the compounds of the present invention include antagonists (and/or inverse agonists) of the H4 receptor, for example, the compounds disclosed in Jablonowski et al., J. Med. Chem. 46:3957-3960 (2003).

The invention thus provides, in a further aspect, a combination comprising a compound of the invention together with a PDE4 inhibitor.

The invention thus provides, in a further aspect, a combination comprising a compound of the invention together with a β₂-adrenoreceptor agonist.

The invention thus provides, in another aspect, a combination comprising a compound of the invention together with a corticosteroid.

The invention thus provides, in another aspect, a combination comprising a compound of the invention together with another non-steroidal GR agonist.

The invention thus provides, in a further aspect, a combination comprising a compound of the invention together with an anticholinergic.

The invention thus provides, in a further aspect, a combination comprising a compound of the invention together with an antihistamine.

The invention thus provides, in a further aspect, a combination comprising a compound of the invention together with a PDE4 inhibitor and a β₂-adrenoreceptor agonist.

The invention thus provides, in a further aspect, a combination comprising a compound of the invention together with an anticholinergic and a PDE-4 inhibitor.

The combinations referred to above may conveniently be presented for use in the form of a pharmaceutical formulation and thus pharmaceutical formulations comprising a combination as defined above together with a pharmaceutically acceptable diluent or carrier represent a further aspect of the invention.

The individual compounds of such combinations may be administered either sequentially or simultaneously in separate or combined pharmaceutical formulations. In one embodiment, they may be administered simultaneously in a combined pharmaceutical formulation. Appropriate doses of known therapeutic agents will be readily appreciated by those skilled in the art.

The invention thus provides, in a further aspect, a pharmaceutical composition comprising a combination of a compound of the invention together with another therapeutically active agent.

The invention thus provides, in a further aspect, a pharmaceutical composition comprising a combination of a compound of the invention together with a PDE4 inhibitor.

The invention thus provides, in a further aspect, a pharmaceutical composition comprising a combination of a compound of the invention together with a β₂-adrenoreceptor agonist.

The invention thus provides, in a further aspect, a pharmaceutical composition comprising a combination of a compound of the invention together with a corticosteroid.

The invention thus provides, in a further aspect, a pharmaceutical composition comprising a combination of a compound of the invention together with another non-steroidal GR agonist.

The invention thus provides, in a further aspect, a pharmaceutical composition comprising a combination of a compound of the invention together with an anticholinergic.

The invention thus provides, in a further aspect, a pharmaceutical composition comprising a combination of a compound of the invention together with an antihistamine.

The invention thus provides, in a further aspect, a pharmaceutical composition comprising a compound of the invention together with a PDE4 inhibitor and a β₂-adrenoreceptor agonist.

The invention thus provides, in a further aspect, a pharmaceutical composition comprising a compound of the invention together with an anticholinergic and a PDE4 inhibitor

The present invention also provides a process for the preparation of compounds of formula (I) comprising reaction of an epoxide of formula (II)

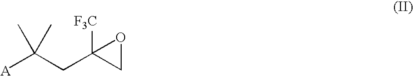

wherein A is as defined above for the compounds of formula (I),
with a 4-amino-1-arylpyrazolopyrimidine of formula (III)

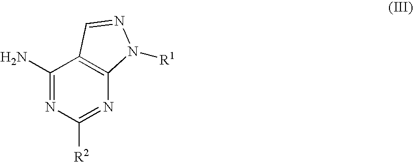

wherein R¹ and R² are as defined above for the compounds of formula (I).

The epoxide opening reaction may be performed in a dipolar aprotic solvent such as N,N-dimethylformamide at a non-extreme temperature in the range 0-100° C., most commonly 20° C. (or room temperature) in the presence of a strong base such as potassium tert-butoxide.

Compounds of formula (II) have been described in racemic form in WO04/063163. Compound of formula (II) wherein A represent 5-fluoro-2-methoxy-phenyl has also been described as separate enantiomers in WO05/234250, WO05/040145 and in Bioorg. Med. Chem. Letters. 2006, 16, 654-657.

Compounds of formula (III) are novel and form another aspect of the invention, and may be prepared by reaction of a 1H-pyrazolopyrimidine-4-amine of formula (IV):

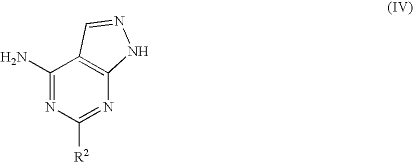

wherein R² is as defined above for the compounds of formula (I),
with an aryl iodide of formula (V)

I—R¹ (V)

wherein R¹ is as defined above for the compounds of formula (I).

The reaction of (IV) with (V) may be performed in the presence of a copper(I) catalyst, such as copper(I) iodide and a weak base such as potassium carbonate or potassium phosphate and an amine ligand such as L-proline, cyclohexanediamine, N,N′-dimethylcyclohexanediamine or N,N′-dimethylethylenediamine in a variety of solvents including toluene, dioxane, N,N-dimethylformamide, N,N-dimethylacetamide and dimethylsulfoxide at a temperature in the range 60-160° C., most typically 110° C. Representative procedures are reported in the literature: Synthesis 2005, 3, 496-499, J. Org. Chem., 2004, 69, 5578-5587 and J. Am. Chem. Soc., 2001, 123, 7727-7729.

Compounds of formula (V) are commercially available.

Compounds of formula (IV) may be prepared by reaction of 4-cyano-5-aminopyrazole (VI):

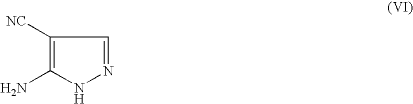

with either acetonitrile or propionitrile in the presence of methanolic ammonia under pressure at high temperature (200° C.). Representative procedures are described in J. Org. Chem., 1961, 26, 4967-4974 (acetonitrile) and Tetrahedron, 1968, 24, 5861-5870 (propionitrile).

An alternative route to compounds of formula (III) involves reaction of a 4-cyano-5-aminopyrazole of formula (VII):

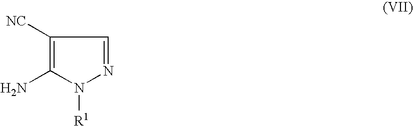

wherein R¹ is as defined above for compounds of formula (I),
with either a nitrile of formula (VIII):

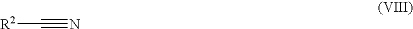

wherein R² is as defined above for compounds of formula (I),
or preferably, an amidine of formula (IX):

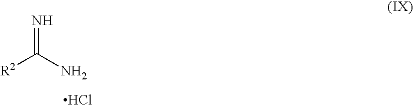

wherein R² is as defined above for compounds of formula (I).

The reaction of (VII) with (VIII) may be performed in the presence of methanolic ammonia under pressure at high temperature, most typically 200° C. Representative procedures are reported in the literature: J. Org. Chem., 1961, 26, 4967-4974, Tetrahedron, 1968, 24, 5861-5870, J. Chem. Soc., Perkin Trans I, 1996, 13, 1545-1552.

The reaction of (VII) with (IX) may be performed in the presence of sodium acetate in a suitable solvent e.g. 2-methoxyethanol or more preferably hexan-1-ol at 150° C.

Compounds of formula (VII) may be prepared by reaction of a hydrazine of formula (X):

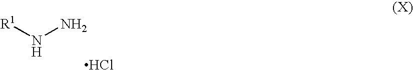

wherein R¹ is as defined above for compounds of formula (I),
with ethoxymethylenemalononitrile in the presence of a mild base e.g. triethylamine in a suitable solvent e.g. ethanol at reflux. A representative procedure is described in the literature: J. Med. Chem., 1996, 39, 1164-1171.

Compounds of formula (I) in which A represents 5-fluoro-2-hydroxy-phenyl may be prepared by reaction of the compounds of formula (I) in which A represents 5-fluoro-2-methoxy-phenyl with, for example, boron tribromide in dichloromethane solution.

Compounds of formula (I) may be prepared in the form of mixtures of enantiomers when mixtures of isomers are used as intermediates in the synthesis. For example, the use of a compound of formula (II) as a racemic mixture of enantiomers will lead to a mixture of enantiomers in the final product. These isomers may, if desired, be separated by conventional methods (eg. HPLC on a chiral column).

Alternatively, separation of isomers may be performed earlier in the synthesis, for example individual isomers of compounds of formula (II) may be employed which may obviate the need to perform a separation of isomers as a final stage in the synthesis. The later process is, in theory, more efficient and is therefore preferred.

In addition, processes for preparing formulations including one or more compounds of formula (I) form an aspect of this invention.

Compositions comprising a compound of the invention also constitute an aspect of the invention.

Solvates of compounds of formula (I), physiologically functional derivatives thereof or salts thereof, which are not physiologically acceptable may be useful as intermediates in the preparation of other compounds of formula (I), physiologically functional derivatives thereof or salts thereof.

Compounds of the invention may be expected to demonstrate good anti-inflammatory properties, with predictable pharmacokinetic and pharmacodynamic behaviour. They also may be expected to have an attractive side-effect profile, demonstrated, for example, by increased selectivity for the glucocorticoid receptor over the progesterone receptor and are expected to be compatible with a convenient regime of treatment in human patients.

The invention will now be illustrated by way of the following non-limiting examples.

### EXAMPLES

### Synthetic Experimental

### Abbreviations

| | |
|---|---|
| DMSO | Dimethylsulphoxide |
| NMR | Nuclear magnetic resonance |
| LCMS | Liquid chromatography/mass spectrometry |
| MeCN | Acetonitrile |
| Me | Methyl |
| Et | Ethyl |

Chromatographic purification was performed using pre-packed Bond Elut silica gel cartridges available commercially from Varian.

### NMR

¹H NMR spectra were recorded in DMSO-d₆ on a Bruker DPX 400 working at 400 MHz. The internal standard used was either tetramethylsilane or the residual protonated solvent at 2.50 ppm for DMSO-d₆.

### Mass Directed Autopreparative HPLC

Autopreparative HPLC was carried out using a Waters 600 gradient pump, Waters 2767 inject/collector, Waters Reagent Manager, Micromass ZMD mass spectrometer, Gilson Aspec waste collector and Gilson 115 post-fraction UV detector. The column used was typically a Supelco LCABZ++ column with dimension of 20 mm internal diameter by 100 mm in length. The stationary phase particle size is 5 μm. The flow rate was 20 ml/min and the runtime was 15 minutes, which comprises a 10-minute gradient followed by a 5 minute column flush and re-equilibration step.

Solvent A: Aqueous solvent=water+0.1% formic acid.

Solvent B: Organic solvent=MeCN:water 95:5+0.05% formic acid

Specific gradients used were dependent upon the retention time in the analytical system. For 1.5-2.2 min, 0-30% B, 2.0-2.8 min, 5-30% B, 2.5-3.0 min, 15-55% B, 2.8-4.0 min, 30-80% B and 3.8-5.5 min, 50-90% B.

### LCMS System

The LCMS system used was as follows:

| Column: | 3.3 cm × 4.6 mm ID, 3 μm ABZ + PLUS from | | |
|---|---|---|---|
| | Supelco | | |
| Flow Rate: | 3 ml/min | | |
| Injection Volume: | 5 μl | | |
| Temp: | RT | | |
| UV Detection Range: | 215 to 330 nm | | |
| Solvents: | A: 0.1% Formic Acid + 10 mMolar Ammonium | | |
| | Acetate. | | |
| | B: 95% Acetonitrile + 0.05% Formic Acid | | |
| | Time | A % | B % |
| Gradient: | 0.00 | 100 | 0 |
| | 0.70 | 100 | 0 |
| | 4.20 | 0 | 100 |
| | 5.30 | 0 | 100 |
| | 5.50 | 100 | 0 |

### Intermediate 1: 6-Methyl-1H-pyrazolo[3,4-d]pyrimidin-4-amine

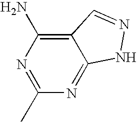

3-Amino-4-pyrazolecarbonitrile (5 g, 46 mmol) and anhydrous acetonitrile (36 ml, 0.69 mol) in 2M ammonia/methanol (100 ml) were stirred and heated to 200° C. (400 psi) in a steel pressure vessel for 48 hours. The reaction mixture was cooled and the precipitate was filtered, washed with methanol followed by ethyl acetate and finally ether and then dried in vacuo to yield the title compound (3.05 g).

LCMS: t_{RET}=0.62 min; MH⁺=150

### Intermediate 2: 6-Ethyl-1H-pyrazolo[3,4-d]pyrimidin-4-amine

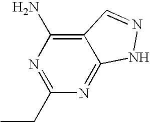

Prepared similarly to Intermediate 1 from 3-amino-4-pyrazolecarbonitrile and propionitrile. LCMS: t_{RET}=0.84 min; MH⁺=164

### Intermediate 3: 6-Methyl-1-phenyl-1H-pyrazolo[3,4-d]pyrimidin-4-amine

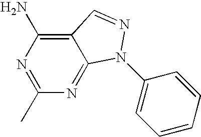

### Method A

To a mixture of 6-methyl-1H-pyrazolo[3,4-d]pyrimidin-4-amine (104 mg, 0.7 mmol), copper(I) iodide (27 mg, 0.14 mmol), potassium carbonate (193 mg, 1.4 mmol) and N,N′-dimethylcyclohexane-1,2-diamine (40 mg, 0.28 mmol) in a reactivial was added a solution of iodobenzene (171 mg, 0.84 mmol) in N,N-dimethylformamide (4 ml). The reactivial was sealed, transferred to a pre-heated heating block (110° C.) and stirred overnight. The reaction mixture was then diluted with dichloromethane (5 ml), filtered through a phase separator cartridge (washed through with 3×10 ml dichloromethane aliquots) and the filtrate evaporated in vacuo. The crude product was purified on a 20 g silica Bond Elut cartridge using a 0-10% methanol in ethyl acetate gradient over 30 mins to give the title compound (30 mg).

¹H-NMR: (DMSO-d₆, 400 MHz) δ 8.28 (s, 1H), 8.21 (dd, J=8.5, 1.0 Hz, 2H), 7.84 (br. s, 1H), 7.70 (br. s, 1H), 7.53 (dd, J=8.5, 7.5 Hz, 2H), 7.32 (tt, J=7.5, 1.0 Hz, 1H), 2.45 (s, 3H)

### Method B

5-Amino-1-phenyl-1H-pyrazole-4-carbonitrile (which may be prepared as described in J. Med. Chem., 1996, 39, 1164-1171) (11.04 g, 60 mmol) and acetonitrile (91 ml, 1.73 mol) in 2M ammonia/methanol (244 ml) were stirred and heated to 200° C. in a steel pressure vessel for 45 hours. The reaction mixture was cooled and the crystalline product was filtered, washed with methanol and dried in vacuo at 40° C. to give the title compound (8.87 g).

LCMS: t_{RET}=2.26 min; MH⁺=226

### Intermediate 4: 1-(2-Fluorophenyl)-6-methyl-1H-pyrazolo[3,4-d]pyrimidin-4-amine

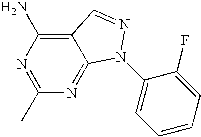

To a mixture of 6-methyl-1H-pyrazolo[3,4-d]pyrimidin-4-amine (104 mg, 0.7 mmol), copper(I) iodide (27 mg, 0.14 mmol), potassium carbonate (193 mg, 1.4 mmol) and N,N′-dimethylcyclohexane-1,2-diamine (40 mg, 0.28 mmol) in a reactivial was added a solution of 1-fluoro-2-iodobenzene (175 mg, 0.79 mmol) in N,N-dimethylformamide (4 ml). The reactivial was sealed, transferred to a pre-heated heating block (110° C.) and stirred overnight. The reaction mixture was then filtered through a phase separator cartridge (washed through with ethyl acetate) and the filtrate evaporated in vacuo. The crude product was purified on a 20 g silica Bond Elut cartridge using a 0-10% methanol in ethyl acetate gradient over 30 mins to give the title compound (14 mg).

LCMS: t_{RET}=2.08 min; MH⁺=244

### Intermediate 5: 1-(4-Fluorophenyl)-6-methyl-1H-pyrazolo[3,4-d]pyrimidin-4-amine

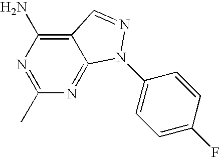

### Method A

Prepared similarly to Intermediate 3 from 6-methyl-1H-pyrazolo[3,4-d]pyrimidin-4-amine and 1-fluoro-4-iodobenzene. The crude product was purified on a 20 g silica Bond Elut cartridge using a 100% dichloromethane to 100% ethyl acetate gradient over 40 mins to give the title compound.

LCMS: t_{RET}=2.34 min; MH⁺=244

### Method B

5-Amino-1-(4-fluorophenyl)-1H-pyrazole-4-carbonitrile (which may be prepared as described in J. Med. Chem., 1991, 34, 2892-2898) (7.45 g, 37 mmol) and acetonitrile (56 ml, 1.07 mol) in 2M ammonia/methanol (150 ml) were stirred and heated to 200° C. in a steel pressure vessel for 45 hours. The reaction mixture was cooled and the precipitate was filtered, washed with methanol and dissolved in ethyl acetate. The ethyl acetate solution was filtered in order to remove a small amount of insoluble solid and then concentrated in vacuo until appearance of precipitate. The ethyl acetate suspension was diluted with an equal volume of chloroform and the solid filtered and washed with 1:1 chloroform/40:60 petroleum ether to give the title compound (4.37 g).

LCMS: t_{RET}=2.42 min; MH⁺=244

A further 1.3 g of title compound was obtained from the acetonitrile/2M ammonia in methanol mother liquor using a similar work-up.

### Intermediate 6: 1-(2,4-Difluorophenyl)-6-methyl-1H-pyrazolo[3,4-d]pyrimidin-4-amine

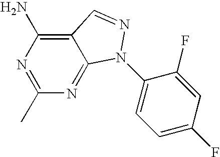

### Method A

Prepared similarly to Intermediate 4 from 6-methyl-1H-pyrazolo[3,4-d]pyrimidin-4-amine and 2,4-difluoro-1-iodobenzene.

LCMS: t_{RET}=2.18 min; MH⁺=262

### Method B

5-Amino-1-(2,4-difluorophenyl)-1H-pyrazole-4-carbonitrile (1.09 g, 5.2 mmol) and anhydrous acetonitrile (6.3 ml, 0.12 mol) in 2M ammonia/methanol (21 ml) were stirred and heated to 200° C. (420 psi) in a steel pressure vessel for 24 hours. The reaction mixture was cooled and evaporated in vacuo. The crude product was preabsorbed onto Florosil (12 g) and purified on 4×50 g silica Bond Elut cartridges using a 0-10% methanol in ethyl acetate gradient to give the title compound (466 mg).

LCMS: t_{RET}=2.24 min; MH⁺=262

### Method C

A suspension of 5-amino-1-(2,4-difluorophenyl)-1H-pyrazole-4-carbonitrile (6.6 g, 30 mmol), acetamidine hydrochloride (11.4 g, 120 mmol) and sodium acetate (9.9 g, 120 mmol) in hexan-1-ol (65 ml) was stirred and heated at 150° C. for 22 hours. The reaction was then cooled and the solid filtered, washed with hexan-1-ol (50 ml) followed by diethylether (2×25 ml) followed by water (2×25 ml) and dried at 50° C. in vacuo overnight to give the title compound (2.0 g).

¹H-NMR: (DMSO-d₆, 400 MHz) δ 8.28 (s, 1H), 7.83 (br. s, 2H), 7.68 (td, J=8.5, 6.0 Hz, 1H), 7.56 (td, J=9.5, 2.5 Hz, 1H), 7.29 (td, J=8.5, 1.5 Hz, 1H), 2.34 (s, 3H)

The hexanol/diethylether filtrate was extracted with 1M hydrochloric acid. The acidic aqueous layer was separated, backwashed with ethyl acetate, adjusted to pH 12 with aqueous sodium hydroxide solution and the resulting solid filtered, washed with water and dried at 50° C. in vacuo overnight to give the title compound (1.38 g).

### Intermediate 7: 6-Methyl-1-(3-pyridinyl)-1H-pyrazolo[3,4-d]pyrimidin-4-amine

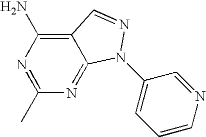

Prepared similarly to Intermediate 4 from 6-methyl-1H-pyrazolo[3,4-d]pyrimidin-4-amine and 3-iodopyridine.

LCMS: t_{RET}=1.94 min; MH⁺=227

### Intermediate 8: 6-Methyl-1-(4-pyridinyl)-1H-pyrazolo[3,4-d]pyrimidin-4-amine

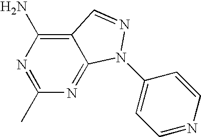

Prepared similarly to Intermediate 3 from 6-methyl-1H-pyrazolo[3,4-d]pyrimidin-4-amine and 4-iodopyridine.

LCMS: t_{RET}=1.87 min; MH⁺=227

### Intermediate 9: 6-Methyl-1-(3-thienyl)-1H-pyrazolo[3,4-d]pyrimidin-4-amine

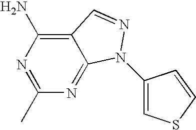

Prepared similarly to Intermediate 3 from 6-methyl-1H-pyrazolo[3,4-d]pyrimidin-4-amine and 3-iodothiophene.

LCMS: t_{RET}=2.31 min; MH⁺=232

### Intermediate 10: 1-(6-Fluoro-3-pyridinyl)-6-methyl-1H-pyrazolo[3,4-d]pyrimidin-4-amine

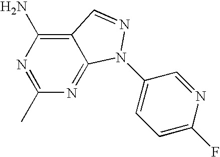

Prepared similarly to Intermediate 3 from 6-methyl-1H-pyrazolo[3,4-d]pyrimidin-4-amine and 2-fluoro-5-iodopyridine. The crude product was purified on a 20 g silica Bond Elut cartridge using a 100% dichloromethane to 100% ethyl acetate gradient over 40 mins to give the title compound.

LCMS: t_{RET}=2.16 min; MH⁺=245

### Intermediate 11: 4-(4-Amino-6-methyl-1H-pyrazolo[3,4-d]pyrimidin-1-yl)benzonitrile

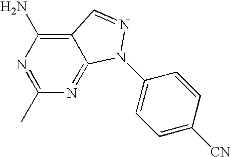

Prepared similarly to Intermediate 4 from 6-methyl-1H-pyrazolo[3,4-d]pyrimidin-4-amine and 4-iodobenzonitrile.

LCMS: t_{RET}=2.69 min; MH⁺=251

### Intermediate 12: Ethyl 3-(4-amino-6-methyl-1H-pyrazolo[3,4-d]pyrimidin-1-yl)benzoate

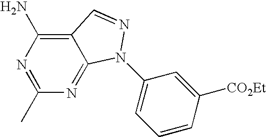

Prepared similarly to Intermediate 3 from 6-methyl-1H-pyrazolo[3,4-d]pyrimidin-4-amine and ethyl 3-iodobenzoate. The crude product was purified on a 20 g silica Bond Elut cartridge using a 100% dichloromethane to 100% ethyl acetate gradient over 40 mins to give the title compound.

LCMS: t_{RET}=2.73 min; MH⁺=298

### Intermediate 13: Methyl 3-(4-amino-6-methyl-1H-pyrazolo[3,4-d]pyrimidin-1-yl)benzoate

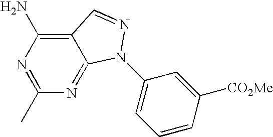

Prepared similarly to Intermediate 3 from 6-methyl-1H-pyrazolo[3,4-d]pyrimidin-4-amine and methyl 3-iodobenzoate.

LCMS: t_{RET}=2.41 min; MH⁺=284

### Intermediate 14: 6-Ethyl-1-phenyl-1H-pyrazolo[3,4-d]pyrimidin-4-amine

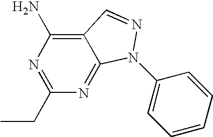

To a mixture of 6-ethyl-1H-pyrazolo[3,4-d]pyrimidin-4-amine (114 mg, 0.7 mmol), copper(I) iodide (27 mg, 0.14 mmol), potassium carbonate (193 mg, 1.4 mmol) and N,N′-dimethylcyclohexane-1,2-diamine (40 mg, 0.28 mmol) in a reactivial was added a solution of iodobenzene (171 mg, 0.84 mmol) in N,N-dimethylformamide (4 ml). The reactivial was sealed, transferred to a pre-heated heating block (110° C.) and stirred overnight. The reaction was then diluted with dichloromethane (5 ml), filtered through a phase separator cartridge (washed through with 3×10 ml dichloromethane aliquots) and the filtrate evaporated in vacuo. The crude product was purified on a 20 g silica Bond Elut cartridge using a 100% dichloromethane to 100% ethyl acetate gradient over 40 mins to give the title compound (78 mg).

LCMS: t_{RET}=2.65 min; MH⁺=240

### Intermediate 15: 6-Ethyl-1-(2-fluorophenyl)-1H-pyrazolo[3,4-d]pyrimidin-4-amine

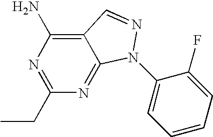

Prepared similarly to Intermediate 14 from 6-ethyl-1H-pyrazolo[3,4-d]pyrimidin-4-amine and 1-fluoro-2-iodobenzene. The crude product was preabsorbed onto silica and purified on a 20 g silica Bond Elut cartridge using a 100% dichloromethane to 100% ethyl acetate gradient over 40 mins to give the title compound.

LCMS: t_{RET}=2.11 min; MH⁺=258

### Intermediate 16: 6-Ethyl-1-(4-fluorophenyl)-1H-pyrazolo[3,4-d]pyrimidin-4-amine

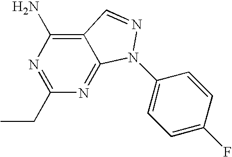

To a mixture of 6-ethyl-1H-pyrazolo[3,4-d]pyrimidin-4-amine (50 mg, 0.31 mmol), copper (I) iodide (10 mg, 0.07 mmol), potassium carbonate (85 mg, 0.61 mmol) and N,N′-dimethylcyclohexane-1,2-diamine (18 mg, 0.12 mmol) in a reactivial was added a solution of 1-fluoro-4-iodobenzene (82 mg, 0.37 mmol) in N,N-dimethylformamide (2 ml). The reactivial was sealed, transferred to a pre-heated heating block (110° C.) and stirred overnight. The reaction was then partitioned between water and dichloromethane, the organic layer was separated and the aqueous phase was back-extracted with further dichloromethane. The combined organic layers were dried over sodium sulphate, filtered and evaporated in vacuo. The crude product was purified by mass-directed autopreparation. The appropriate fractions were neutralised using aqueous sodium hydrogen carbonate solution, extracted into dichloromethane and evaporated to give the title compound (44 mg).

LCMS: t_{RET}=2.73 min; MH⁺=258

### Intermediate 17: 1-(2,4-Difluorophenyl)-6-ethyl-1H-pyrazolo[3,4-d]pyrimidin-4-amine

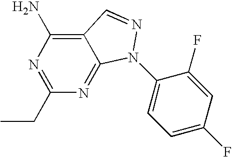

Prepared similarly to Intermediate 14 from 6-ethyl-1H-pyrazolo[3,4-d]pyrimidin-4-amine and 2,4-difluoro-1-iodobenzene. The crude product was preabsorbed onto silica and purified on a 20 g silica Bond Elut cartridge using a 100% dichloromethane to 100% ethyl acetate gradient over 40 mins to give the title compound.

LCMS: t_{RET}=2.36 min; MH⁺=276

### Intermediate 18: 6-Ethyl-1-(3-pyridinyl)-1H-pyrazolo[3,4-d]pyrimidin-4-amine

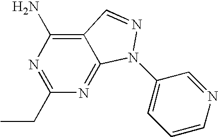

Prepared similarly to Intermediate 14 from 6-ethyl-1H-pyrazolo[3,4-d]pyrimidin-4-amine and 3-iodopyridine. The crude product was preabsorbed onto silica and purified on a 20 g silica Bond Elut cartridge using a 100% dichloromethane to 100% ethyl acetate gradient over 40 mins to give the title compound.

LCMS: t_{RET}=2.20 min; MH⁺=241

### Intermediate 19: 6-Ethyl-1-(4-pyridinyl)-1H-pyrazolo[3,4-d]pyrimidin-4-amine

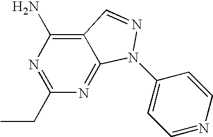

Prepared similarly to Intermediate 14 from 6-ethyl-1H-pyrazolo[3,4-d]pyrimidin-4-amine and 4-iodopyridine. The crude product was preabsorbed onto silica and purified on a 20 g silica Bond Elut cartridge using a 100% dichloromethane to 100% ethyl acetate gradient over 40 mins to give the title compound.

LCMS: t_{RET}=2.08 min; MH⁺=241

### Intermediate 20: 6-Ethyl-1-(6-fluoro-3-pyridinyl)-1H-pyrazolo[3,4-d]pyrimidin-4-amine

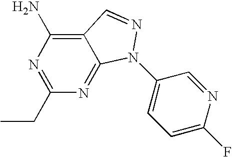

Prepared similarly to Intermediate 14 from 6-ethyl-1H-pyrazolo[3,4-d]pyrimidin-4-amine and 2-fluoro-5-iodopyridine.

LCMS: t_{RET}=2.41 min; MH⁺=259

### Intermediate 21: 6-Ethyl-1-(3-thienyl)-1H-pyrazolo[3,4-d]pyrimidin-4-amine

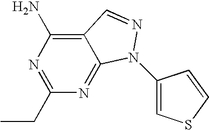

Prepared similarly to Intermediate 14 from 6-ethyl-1H-pyrazolo[3,4-d]pyrimidin-4-amine and 3-iodothiophene.

LCMS: t_{RET}=2.60 min; MH⁺=246

### Intermediate 22: 5-Amino-1-(2,4-difluorophenyl)-1H-pyrazole-4-carbonitrile

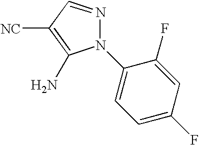

A mixture of ethoxymethylenemalononitrile (57.6 g, 0.47 mol), 2,4-difluorophenylhydrazine hydrochloride (85.3 g, 0.47 mol) and triethylamine (57.7 g, 0.57 mol) in ethanol (1000 ml) was stirred and heated at reflux for 1 hour and then cooled in an ice bath. Diethylether (750 ml) was added and the precipitated solid was filtered, washed with diethylether (2×200 ml) and dried at room temperature in vacuo overnight. The crude product contained triethylamine hydrochloride and was therefore suspended in water (400 ml), stirred at room temperature for 10 mins, filtered, washed with water (2×150 ml) and dried at 50° C. in vacuo overnight to yield the title compound (41.8 g). The ethanol/diethylether mother liquor was concentrated in vacuo to low volume and the resulting solid was filtered, washed with diethylether followed by water and dried at 50° C. in vacuo overnight to yield the title compound (41.4 g). The two batches of material were combined, suspended in diethylether, filtered, washed with diethylether and dried at room temperature in vacuo to yield the title compound as a light brown solid (79.6 g).

¹H-NMR: (DMSO-d₆, 400 MHz) δ 7.78 (s, 1H), 7.58 (td, J=8.6, 6.0 Hz, 1H), 7.53 (td, J=9.5, 2.5 Hz, 1H), 7.25 (td, J=8.5, 2.0 Hz, 1H), 6.79 (br. s, 2H)

### Intermediate 23: 3-(4-Amino-6-methyl-1H-pyrazolo[3,4-d]pyrimidin-1-yl)benzonitrile

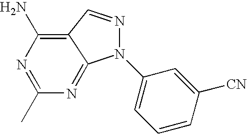

Prepared similarly to Intermediate 3 from 6-methyl-1H-pyrazolo[3,4-d]pyrimidin-4-amine and 3-iodobenzonitrile. The crude product was purified on a 20 g silica Bond Elut cartridge using a 100% dichloromethane to 100% ethyl acetate gradient over 40 mins to give the title compound.

LCMS: t_{RET}=2.52 min; MH⁺=251

### Intermediate 24: 1-(2-Fluoro-3-pyridinyl)-6-methyl-1H-pyrazolo[3,4-d]pyrimidin-4-amine

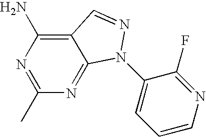

Prepared similarly to Intermediate 3 from 6-methyl-1H-pyrazolo[3,4-d]pyrimidin-4-amine and 2-fluoro-3-iodopyridine. The crude product was purified by mass-directed autopreparation. The appropriate fractions were neutralised using aqueous sodium hydrogen carbonate solution, extracted into dichloromethane and evaporated to give the title compound.

LCMS: t_{RET}=1.89 min; MH⁺=245

### Intermediate 25: 3-(4-Amino-6-ethyl-1H-pyrazolo[3,4-d]pyrimidin-1-yl)benzonitrile

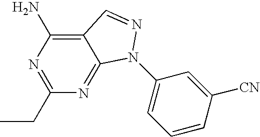

Prepared similarly to Intermediate 14 from 6-ethyl-1H-pyrazolo[3,4-d]pyrimidin-4-amine and 3-iodobenzonitrile.

LCMS: t_{RET}=2.95 min; MH⁺=265

### Intermediate 26: Methyl 3-(4-amino-6-ethyl-1H-pyrazolo[3,4-d]pyrimidin-1-yl)benzoate

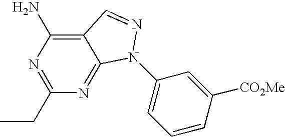

Prepared similarly to Intermediate 14 from 6-ethyl-1H-pyrazolo[3,4-d]pyrimidin-4-amine and methyl 3-iodobenzoate.

LCMS: t_{RET}=2.91 min; MH⁺=298

### Example 1

### 4-(2,3-Dihydro-1-benzofuran-7-yl)-1,1,1-trifluoro-4-methyl-2-{[(6-methyl-1-phenyl-1H-pyrazolo[3,4-d]pyrimidin-4-yl)amino]methyl}-2-pentanol

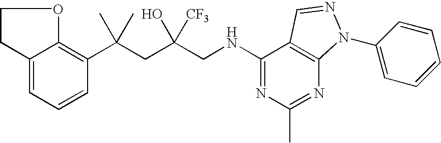

To a solution of 6-methyl-1-phenyl-1H-pyrazolo[3,4-d]pyrimidin-4-amine (15.1 mg, 0.067 mmol) in N,N-dimethylformamide (0.4 ml) was added racemic 7-{1,1-dimethyl-2-[2-(trifluoromethyl)-2-oxiranyl]ethyl}-2,3-dihydro-1-benzofuran (which may be prepared as described in WO 04/063163) (19.2 mg, 0.067 mmol) followed by potassium t-butoxide (7.5 mg, 0.067 mmol). The reaction was stirred at room temperature under nitrogen overnight and then partitioned between ethyl acetate (20 ml) and water (20 ml). The organic layer was separated and the aqueous phase was back-extracted with further ethyl acetate. The combined organic layers were washed with aqueous sodium hydrogen carbonate solution (×2) followed by water and finally brine, filtered through a hydrophobic frit and evaporated in vacuo. The crude product was purified by mass-directed autopreparation to give the title compound (10.8 mg).

LCMS: t_{RET}=4.23 min; MH⁺=512

### Example 2

### 4-(2,3-Dihydro-1-benzofuran-7-yl)-1,1,1-trifluoro-2-({[1-(2-fluorophenyl)-6-methyl-1H-pyrazolo[3,4-d]pyrimidin-4-yl]amino}methyl)-4-methyl-2-pentanol

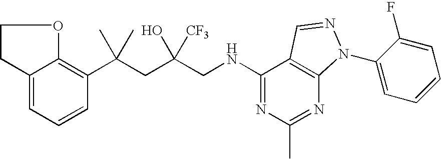

To 1-(2-fluorophenyl)-6-methyl-1H-pyrazolo[3,4-d]pyrimidin-4-amine (7.1 mg, 0.029 mmol) was added a solution of racemic 7-{1,1-dimethyl-2-[2-(trifluoromethyl)-2-oxiranyl]ethyl}-2,3-dihydro-1-benzofuran (9.2 mg, 0.032 mmol) in anhydrous N,N-dimethylformamide (0.23 ml) followed by potassium t-butoxide (3.7 mg, 0.033 mmol). The reaction was stirred at room temperature under nitrogen overnight. The crude reaction mixture was purified by custom purification (see below).

| Column: | Supelcosil ABZ + plus 100 × 21.2 mm, 5 um | |
|---|---|---|
| Eluent: | Organic - 95% MeCN + 0.05% Formic Acid | |
| | Aqueous - Water + 0.1% Formic Acid | |
| | Time (mins) | % Organic |
| Gradient: | 0 | 60 |
| | 1 | 60 |
| | 25 | 80 |
| | 27 | 100 |
| | 28.5 | 100 |
| | 29 | 60 |
| | 30 | 60 |

| | |
|---|---|
| Run Time: | 30 minutes |
| Flow Rate: | 20 ml/minute |

The product fractions were partitioned between dichloromethane and aqueous sodium hydrogen carbonate solution. The organic layer was separated, washed with water followed by brine, filtered through a hydrophobic frit and evaporated in vacuo to give the title compound (6 mg).

LCMS: t_{RET}=3.93 min; MH⁺=530

### Example 3

### 4-(2,3-Dihydro-1-benzofuran-7-yl)-1,1,1-trifluoro-2-({[1-(4-fluorophenyl)-6-methyl-1H-pyrazolo[3,4-d]pyrimidin-4-yl]amino}methyl)-4-methyl-2-pentanol

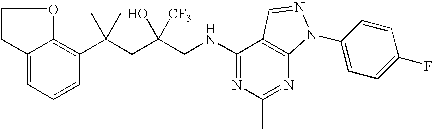

Prepared similarly to Example 2 from racemic 7-{1,1-dimethyl-2-[2-(trifluoromethyl)-2-oxiranyl]ethyl}-2,3-dihydro-1-benzofuran and 1-(4-fluorophenyl)-6-methyl-1H-pyrazolo[3,4-d]pyrimidin-4-amine. The crude reaction mixture was purified by mass-directed autopreparation to give the title compound.

LCMS: t_{RET}=4.26 min; MH⁺=530

### Example 4

### 2-({[1-(2,4-Difluorophenyl)-6-methyl-1H-pyrazolo[3,4-d]pyrimidin-4-yl]amino}methyl)-4-(2,3-dihydro-1-benzofuran-7-yl)-1,1,1-trifluoro-4-methyl-2-pentanol

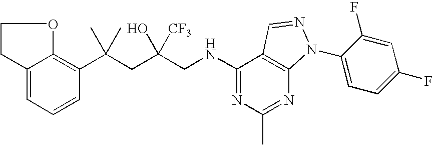

Prepared similarly to Example 2 from racemic 7-{1,1-dimethyl-2-[2-(trifluoromethyl)-2-oxiranyl]ethyl}-2,3-dihydro-1-benzofuran and 1-(2,4-difluorophenyl)-6-methyl-1H-pyrazolo[3,4-d]pyrimidin-4-amine. The crude reaction mixture was purified by mass-directed autopreparation. The appropriate fractions were neutralised using aqueous sodium hydrogen carbonate solution and extracted into dichloromethane. The organic layer was separated and the aqueous phase was back-extracted with further dichloromethane. The combined organic layers were washed with water followed by brine, filtered through a hydrophobic frit and evaporated in vacuo to give the title compound (37 mg).

LCMS: t_{RET}=3.98 min; MH⁺=548

### Example 5

### 4-(2,3-Dihydro-1-benzofuran-7-yl)-2-{[(6-ethyl-1-phenyl-1H-pyrazolo[3,4-d]pyrimidin-4-yl)amino]methyl}-1,1,1-trifluoro-4-methyl-2-pentanol

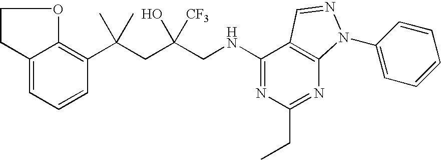

Prepared similarly to Example 2 from racemic 7-{1,1-dimethyl-2-[2-(trifluoromethyl)-2-oxiranyl]ethyl}-2,3-dihydro-1-benzofuran and 6-ethyl-1-phenyl-1H-pyrazolo[3,4-d]pyrimidin-4-amine. The crude reaction mixture was purified by mass-directed autopreparation to give the title compound.

LCMS: t_{RET}=4.40 min; MH⁺=526

### Example 6

### 4-(2,3-Dihydro-1-benzofuran-7-yl)-2-({[6-ethyl-1-(2-fluorophenyl)-1H-pyrazolo[3,4-d]pyrimidin-4-yl]amino}methyl)-1,1,1-trifluoro-4-methyl-2-pentanol

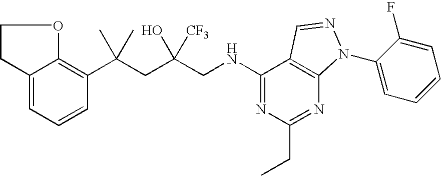

Prepared similarly to Example 2 from racemic 7-{1,1-dimethyl-2-[2-(trifluoromethyl)-2-oxiranyl]ethyl}-2,3-dihydro-1-benzofuran and 6-ethyl-1-(2-fluorophenyl)-1H-pyrazolo[3,4-d]pyrimidin-4-amine. The crude reaction mixture was purified by mass-directed autopreparation to give the title compound.

LCMS: t_{RET}=4.06 min; MH⁺=544

### Example 7

### 4-(2,3-Dihydro-1-benzofuran-7-yl)-2-({[6-ethyl-1-(4-fluorophenyl)-1H-pyrazolo[3,4-d]pyrimidin-4-yl]amino}methyl)-1,1-trifluoro-4-methyl-2-pentanol

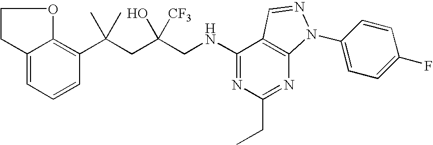

Prepared similarly to Example 2 from racemic 7-{1,1-dimethyl-2-[2-(trifluoromethyl)-2-oxiranyl]ethyl}-2,3-dihydro-1-benzofuran and 6-ethyl-1-(4-fluorophenyl)-1H-pyrazolo[3,4-d]pyrimidin-4-amine. The crude reaction mixture was purified by mass-directed autopreparation to give the title compound.

LCMS: t_{RET}=4.42 min; MH⁺=544

### Example 8

### 2-({[1-(2,4-Difluorophenyl)-6-ethyl-1H-pyrazolo[3,4-d]pyrimidin-4-yl]amino}methyl)-4-(2,3-dihydro-1-benzofuran-7-yl)-1,1,1-trifluoro-4-methyl-2-pentanol

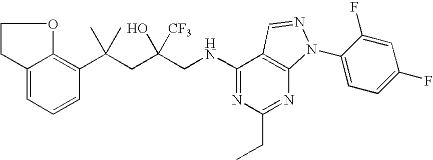

Prepared similarly to Example 2 from racemic 7-{1,1-dimethyl-2-[2-(trifluoromethyl)-2-oxiranyl]ethyl}-2,3-dihydro-1-benzofuran and 1-(2,4-difluorophenyl)-6-ethyl-1H-pyrazolo[3,4-d]pyrimidin-4-amine. The crude reaction mixture was purified by mass-directed autopreparation to give the title compound.

LCMS: t_{RET}=4.09 min; MH⁺=562

### Example 9

### 4-(2,3-Dihydro-1-benzofuran-7-yl)-2-({[6-ethyl-1-(3-pyridinyl)-1H-pyrazolo[3,4-d]pyrimidin-4-yl]amino}methyl)-1,1,1-trifluoro-4-methyl-2-pentanol

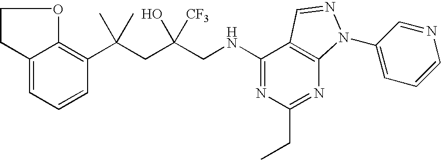

Prepared similarly to Example 2 from racemic 7-{1,1-dimethyl-2-[2-(trifluoromethyl)-2-oxiranyl]ethyl}-2,3-dihydro-1-benzofuran and 6-ethyl-1-(3-pyridinyl)-1H-pyrazolo[3,4-d]pyrimidin-4-amine. The crude reaction mixture was purified by mass-directed autopreparation to give the title compound.

LCMS: t_{RET}=4.02 min; MH⁺=527

### Example 10

### 4-(2,3-Dihydro-1-benzofuran-7-yl)-2-({[6-ethyl-1-(4-pyridinyl)-1H-pyrazolo[3,4-d]pyrimidin-4-yl]amino}methyl)-1,1,1-trifluoro-4-methyl-2-pentanol

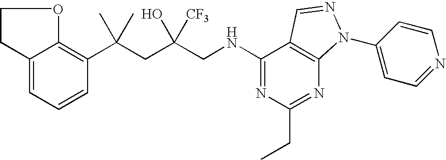

Prepared similarly to Example 2 from racemic 7-{1,1-dimethyl-2-[2-(trifluoromethyl)-2-oxiranyl]ethyl}-2,3-dihydro-1-benzofuran and 6-ethyl-1-(4-pyridinyl)-1H-pyrazolo[3,4-d]pyrimidin-4-amine. The crude reaction mixture was purified by mass-directed autopreparation to give the title compound.

LCMS: t_{RET}=3.99 min; MH⁺=527

### Example 11

### 4-(2,3-Dihydro-1-benzofuran-7-yl)-2-({[6-ethyl-1-(6-fluoro-3-pyridinyl)-1H-pyrazolo[3,4-d]pyrimidin-4-yl]amino}methyl)-1,1,1-trifluoro-4-methyl-2-pentanol

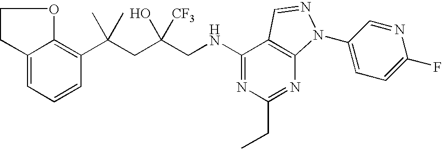

Prepared similarly to Example 2 from racemic 7-{1,1-dimethyl-2-[2-(trifluoromethyl)-2-oxiranyl]ethyl}-2,3-dihydro-1-benzofuran and 6-ethyl-1-(6-fluoro-3-pyridinyl)-1H-pyrazolo[3,4-d]pyrimidin-4-amine. The crude reaction mixture was purified by mass-directed autopreparation to give the title compound.

LCMS: t_{RET}=4.27 min; MH⁺=545

### Example 12

### 4-(2,3-Dihydro-1-benzofuran-7-yl)-2-({[6-ethyl-1-(3-thienyl)-1H-pyrazolo[3,4-d]pyrimidin-4-yl]amino}methyl)-1,1,1-trifluoro-4-methyl-2-pentanol

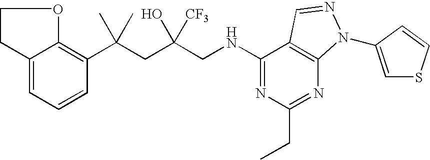

Prepared similarly to Example 2 from racemic 7-{1,1-dimethyl-2-[2-(trifluoromethyl)-2-oxiranyl]ethyl}-2,3-dihydro-1-benzofuran and 6-ethyl-1-(3-thienyl)-1H-pyrazolo[3,4-d]pyrimidin-4-amine. The crude reaction mixture was purified by mass-directed autopreparation to give the title compound.

LCMS: t_{RET}=4.44 min; MH⁺=532

### Example 13

### 1,1,1-Trifluoro-4-[5-fluoro-2-(methyloxy)phenyl]-4-methyl-2-{[(6-methyl-1-phenyl-1H-pyrazolo[3,4-d]pyrimidin-4-yl)amino]methyl}-2-pentanol

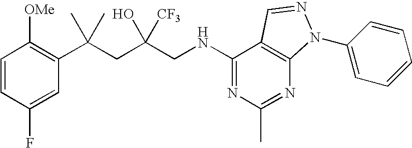

Prepared similarly to Example 2 from racemic 2-{2-[5-fluoro-2-(methyloxy)phenyl]-2-methylpropyl}-2-(trifluoromethyl)oxirane (which may be prepared as described in WO 04/063163) and 6-methyl-1-phenyl-1H-pyrazolo[3,4-d]pyrimidin-4-amine. The crude reaction mixture was purified by mass-directed autopreparation to give the title compound.

LCMS: t_{RET}=4.15 min; MH⁺=518

### Example 14

### 1,1,1-Trifluoro-4-[5-fluoro-2-(methyloxy)phenyl]-2-({[1-(2-fluorophenyl)-6-methyl-1H-pyrazolo[3,4-d]pyrimidin-4-yl]amino}methyl)-4-methyl-2-pentanol

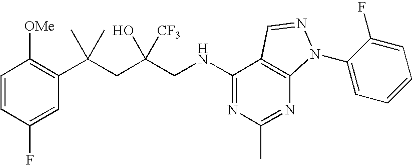

Prepared similarly to Example 2 from racemic 2-{2-[5-fluoro-2-(methyloxy)phenyl]-2-methylpropyl}-2-(trifluoromethyl)oxirane and 1-(2-fluorophenyl)-6-methyl-1H-pyrazolo[3,4-d]pyrimidin-4-amine. The crude reaction mixture was purified by mass-directed autopreparation to give the title compound.

LCMS: t_{RET}=3.89 min; MH⁺=536

The enantiomers were then separated using a 2″×20 cm Chiralcel OD column eluting with 10% ethanol in heptane with a flow rate of 75 ml/min.

### Example 14A

(Enantiomer 1) eluted around 8.9 min and on analytical chiral HPLC (25×0.46 cm Chiralcel OD-H column, 20% ethanol in heptane eluting at 1 ml/min) showed a retention time 4.87 min: LCMS: t_{RET}=3.84 min; MH⁺=536

### Example 14B

(Enantiomer 2) eluted around 13.2 min and on analytical chiral HPLC (25×0.46 cm Chiralcel OD-H column, 20% ethanol in heptane eluting at 1 ml/min) showed a retention time 6.10 min: LCMS: t_{RET}=3.88 min; MH⁺=536

### Example 15

### 1,1,1-Trifluoro-4-[5-fluoro-2-(methyloxy)phenyl]-2-({[1-(4-fluorophenyl)-6-methyl-1H-pyrazolo[3,4-d]pyrimidin-4-yl]amino}methyl)-4-methyl-2-pentanol

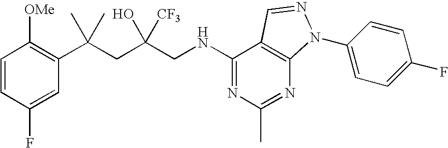

Prepared similarly to Example 2 from racemic 2-{2-[5-fluoro-2-(methyloxy)phenyl]-2-methylpropyl}-2-(trifluoromethyl)oxirane and 1-(4-fluorophenyl)-6-methyl-1H-pyrazolo[3,4-d]pyrimidin-4-amine. The crude reaction mixture was purified by mass-directed autopreparation to give the title compound.

LCMS: t_{RET}=4.22 min; MH⁺=536

### Example 16

### 2-({[1-(2,4-Difluorophenyl)-6-methyl-1H-pyrazolo[3,4-d]pyrimidin-4-yl]amino}methyl)-1,1,1-trifluoro-4-[5-fluoro-2-(methyloxy)phenyl]-4-methyl-2-pentanol

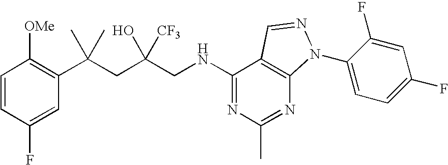

### Method A

1-(2,4-Difluorophenyl)-6-methyl-1H-pyrazolo[3,4-d]pyrimidin-4-amine (411 mg, 1.57 mmol) was dissolved in N,N-dimethylformamide (3 ml) and added to potassium t-butoxide (176 mg, 1.57 mmol) at room temperature under nitrogen. After approximately 30 mins a solution of racemic 2-{2-[5-fluoro-2-(methyloxy)phenyl]-2-methylpropyl}-2-(trifluoromethyl)oxirane (460 mg, 1.57 mmol) in N,N-dimethylformamide (3 ml) was added dropwise. The brown solution was stirred at room temperature for 39 hours and then partitioned between dichloromethane and aqueous ammonium chloride solution. The organic layer was separated and the aqueous phase was back-extracted with further dichloromethane. The combined organic layers were dried over sodium sulphate, filtered and evaporated in vacuo. The crude product was dissolved in ethanol, loaded onto florisil (5 g) and evaporated in vacuo. The absorbed product was purified on a 20 g silica Bond Elut cartridge using a 0-100% ethyl acetate in cyclohexane gradient over 60 mins to give the title compound (705 mg).

LCMS: t_{RET}=3.98 min; MH⁺=554

The enantiomers were then separated using a 2″×20 cm Chiralpak AD column eluting with 2% ethanol in heptane with a flow rate of 75 ml/min.

### Example 16A

(Enantiomer 1, 2R isomer) eluted around 9.8 min and on analytical chiral HPLC (25×0.46 cm Chiralpak AD column, 3% ethanol in heptane eluting at 1 ml/min) showed a retention time 11.2 min: LCMS: t_{RET}=3.98 min; MH⁺=554

### Example 16B

(Enantiomer 2, 2S isomer) eluted around 13.1 min and on analytical chiral HPLC (25×0.46 cm Chiralpak AD column, 3% ethanol in heptane eluting at 1 ml/min) showed a retention time 14.7 min: LCMS: t_{RET}=3.98 min; MH⁺=554

### Method B

Potassium t-butoxide (21.6 g, 192.5 mmol) was added to a stirred suspension of 1-(2,4-difluorophenyl)-6-methyl-1H-pyrazolo[3,4-d]pyrimidin-4-amine (31.3 g, 119.5 mmol) in N,N-dimethylformamide (200 ml) at 20° C. After 30 min a solution of racemic 2-{2-[5-fluoro-2-(methyloxy)phenyl]-2-methylpropyl}-2-(trifluoromethyl)oxirane (28.5 g, 97.5 mmol) in N,N-dimethylformamide (200 ml) was added over 30 min and the mixture stirred for a further 18 hours when aqueous ammonium chloride (25% w/w, 280 ml), ethyl acetate (420 ml) and water (280 ml) were added. The organic phase was separated, combined with a second ethyl acetate extract and evaporated to an oil which was purified by Biotage silica gel chromatography using a 15 to 30% gradient of ethyl acetate in cyclohexane to afford the title compound (44 g). 42.9 g of this racemic material was subjected to enantiomer separation using a Varian SD-2 800 G Prep HPLC system set-up with a 30×10 cm preparative HPLC column packed with Chiralcel OD 20 micron chiral stationary phase. The racemate was dissolved in ethanol (5 volumes) and aliquots containing 1.3 g of material were diluted with n-heptane (9 ml) and immediately injected onto the column which was eluted with 96:4 n-heptane:ethanol at a flow rate of 300 ml/min. Fractions containing enantiomer 1 eluted between 14.75 and 18.75 min and these were combined and evaporated to give Example 16A as an off-white solid (19.4 g)

LCMS: t_{RET}=3.82 min; MH⁺=554. Analytical chiral HPLC using a Chiralcel OD analytical column (250×4.6 mm) eluting with 90:10 n-heptane:ethanol at 0.8 mL/min and a column temperature of 30° C. indicated chiral purity of 99%.

### Example 16A

### (2S)-2-({[1-(2,4-Difluorophenyl)-6-methyl-1H-pyrazolo[3,4-d]pyrimidin-4-yl]amino}methyl)-1,1,1-trifluoro-4-[5-fluoro-2-(methyloxy)phenyl]-4-methyl-2-pentanol

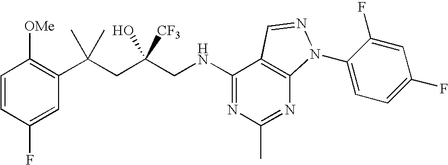

Potassium t-butoxide (26.5 mg, 0.24 mmol) was added to a solution of 1-(2,4-difluorophenyl)-6-methyl-1H-pyrazolo[3,4-d]pyrimidin-4-amine (53.6 mg, 0.205 mmol) and (2S)-2-{2-[5-fluoro-2-(methyloxy)phenyl]-2-methylpropyl}-2-(trifluoromethyl)oxirane ((which may be prepared as described in Bioorg. Med. Chem. Letters. 2006, 16, 654-657) 60 mg, 0.205 mmol) in N,N-dimethylformamide (1.7 ml) and the mixture stirred under nitrogen at room temperature for 48 hours. The mixture was then diluted with methanol, filtered and purified by mass directed autopreparation. Product containing fractions were combined and partitioned between dichloromethane and aqueous sodium bicarbonate. The organic layer was separated, washed successively with water and brine, dried through a hydrophobic frit and evaporated to give the title compound (59.3 mg).

LCMS: t_{RET}=3.82 min; MH⁺=554. Analytical chiral HPLC (25×0.46 cm Chiralcel OD-H column, heptane:EtOH 9:1 eluting at 1 ml/min): t_{RET}=6.3 min. Using this same analytical chiral HPLC system the previously separated enantiomers of Example 16 showed t_{RET}=7.4 min (Example 16A) and 6.3 min (Example 16B).

### Example 17

### 1,1,1-Trifluoro-4-[5-fluoro-2-(methyloxy)phenyl]-4-methyl-2-({[6-methyl-1-(3-pyridinyl)-1H-pyrazolo[3,4-d]pyrimidin-4-yl]amino}methyl)-2-pentanol

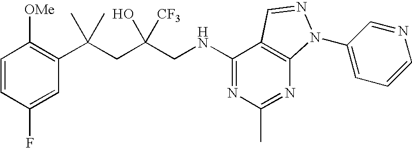

Prepared similarly to Example 2 from racemic 2-{2-[5-fluoro-2-(methyloxy)phenyl]-2-methylpropyl}-2-(trifluoromethyl)oxirane and 6-methyl-1-(3-pyridinyl)-1H-pyrazolo[3,4-d]pyrimidin-4-amine. The crude reaction mixture was purified by custom purification (see below) to give the title compound.

LCMS: t_{RET}=3.87 min; MH⁺=519

| LCMS: | t_{RET} = 3.87 mm; MH⁺ = 519 | |
|---|---|---|
| Column: | Agilent Zorbax SB Phenyl 150 × 21.2 mm ID 7 um | |
| Eluent: | Organic: | 0.05% TFA/MeOH |
| | Aqueous: | 0.1% TFA/water |

| | |
|---|---|
| Gradient: | 71% organic for 25 minutes at 20 ml/min, followed by a |
| | ramp to 100%, followed by re-equilibration prior to |
| | subsequent injection. |
| Run Time: | 30 minute cycle time, two injections per vial |

### Example 18

### 1,1,1-Trifluoro-4-[5-fluoro-2-(methyloxy)phenyl]-4-methyl-2-({[6-methyl-1-(4-pyridinyl)-1H-pyrazolo[3,4-d]pyrimidin-4-yl]amino}methyl)-2-pentanol

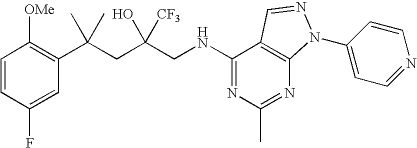

Prepared similarly to Example 2 from racemic 2-{2-[5-fluoro-2-(methyloxy)phenyl]-2-methylpropyl}-2-(trifluoromethyl)oxirane and 6-methyl-1-(4-pyridinyl)-1H-pyrazolo[3,4-d]pyrimidin-4-amine. The crude reaction mixture was purified by mass-directed autopreparation to give the title compound.

LCMS: t_{RET}=3.72 min; MH⁺=519

### Example 19

### 1,1,1-Trifluoro-4-[5-fluoro-2-(methyloxy)phenyl]-4-methyl-2-({[6-methyl-1-(3-thienyl)-1H-pyrazolo[3,4-d]pyrimidin-4-yl]amino}methyl)-2-pentanol

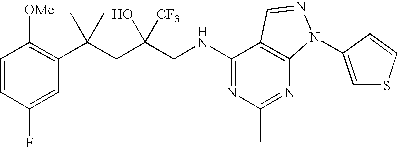

Prepared similarly to Example 2 from racemic 2-{2-[5-fluoro-2-(methyloxy)phenyl]-2-methylpropyl}-2-(trifluoromethyl)oxirane and 6-methyl-1-(3-thienyl)-1H-pyrazolo[3,4-d]pyrimidin-4-amine. The crude reaction mixture was purified by mass-directed autopreparation to give the title compound.

LCMS: t_{RET}=4.19 min; MH⁺=524

### Example 20

### 1,1,1-Trifluoro-4-[5-fluoro-2-(methyloxy)phenyl]-2-({[1-(6-fluoro-3-pyridinyl)-6-methyl-1H-pyrazolo[3,4-d]pyrimidin-4-yl]amino}methyl)-4-methyl-2-pentanol

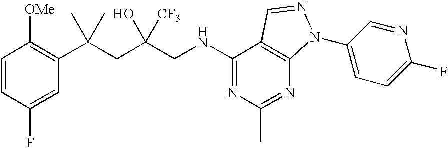

Prepared similarly to Example 2 from racemic 2-{2-[5-fluoro-2-(methyloxy)phenyl]-2-methylpropyl}-2-(trifluoromethyl)oxirane and 1-(6-fluoro-3-pyridinyl)-6-methyl-1H-pyrazolo[3,4-d]pyrimidin-4-amine. The crude reaction mixture was purified by mass-directed autopreparation to give the title compound.

LCMS: t_{RET}=4.08 min; MH⁺=537

### Example 21

### 4-(4-{[4-[5-Fluoro-2-(methyloxy)phenyl]-2-hydroxy-4-methyl-2-(trifluoromethyl)pentyl]amino}-6-methyl-1H-pyrazolo[3,4-d]pyrimidin-1-yl)benzonitrile

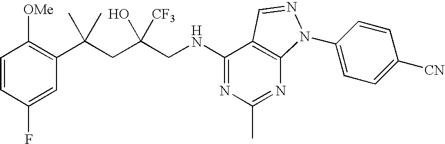

Prepared similarly to Example 2 from racemic 2-{2-[5-fluoro-2-(methyloxy)phenyl]-2-methylpropyl}-2-(trifluoromethyl)oxirane and 4-(4-amino-6-methyl-1H-pyrazolo[3,4-d]pyrimidin-1-yl)benzonitrile. The crude reaction mixture was purified by custom purification (see below) to give the title compound.

LCMS: t_{RET}=4.20 min; MH⁺=543

| Column: | Supelcosil ABZ + plus 100 × 21.2 mm, 5 um | |
|---|---|---|
| Eluent: | Organic - 95% MeCN + 0.05% Formic Acid | |
| | Aqueous - Water + 0.1% Formic Acid | |
| | Time (mins) | % Organic |
| Gradient: | 0 | 70 |
| | 1 | 70 |
| | 12 | 80 |
| | 14 | 99 |
| | 14.8 | 99 |
| | 15 | 70 |

| | |
|---|---|
| Run Time: | 15 minutes |
| Flow Rate: | 20 ml/minute |

### Example 22

### Ethyl 3-(4-{[4-[5-fluoro-2-(methyloxy)phenyl]-2-hydroxy-4-methyl-2-(trifluoromethyl)pentyl]amino}-6-methyl-1H-pyrazolo[3,4-d]pyrimidin-1-yl)benzoate

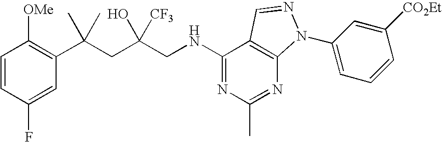

Prepared similarly to Example 2 from racemic 2-{2-[5-fluoro-2-(methyloxy)phenyl]-2-methylpropyl}-2-(trifluoromethyl)oxirane and ethyl 3-(4-amino-6-methyl-1H-pyrazolo[3,4-d]pyrimidin-1-yl)benzoate. The crude reaction mixture was purified by mass-directed autopreparation to give the title compound.

LCMS: t_{RET}=4.35 min; MH⁺=590

### Example 23

### Methyl 3-(4-{[4-[5-fluoro-2-(methyloxy)phenyl]-2-hydroxy-4-methyl-2-(trifluoromethyl)pentyl]amino}-6-methyl-1H-pyrazolo[3,4-d]pyrimidin-1-yl)benzoate

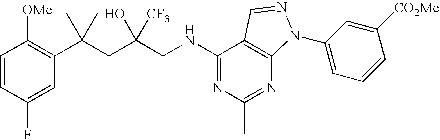

Prepared similarly to Example 2 from racemic 2-{2-[5-fluoro-2-(methyloxy)phenyl]-2-methylpropyl}-2-(trifluoromethyl)oxirane and methyl 3-(4-amino-6-methyl-1H-pyrazolo[3,4-d]pyrimidin-1-yl)benzoate. The crude reaction mixture was purified by mass-directed autopreparation to give the title compound.

LCMS: t_{RET}=4.09 min; MH⁺=576

### Example 24

### 2-({[6-Ethyl-1-(4-fluorophenyl)-1H-pyrazolo[3,4-d]pyrimidin-4-yl]amino}methyl)-1,1,1-trifluoro-4-[5-fluoro-2-(methyloxy)phenyl]-4-methyl-2-pentanol

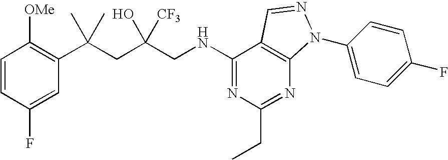

Prepared similarly to Example 2 from racemic 2-{2-[5-fluoro-2-(methyloxy)phenyl]-2-methylpropyl}-2-(trifluoromethyl)oxirane and 6-ethyl-1-(4-fluorophenyl)-1H-pyrazolo[3,4-d]pyrimidin-4-amine. The crude reaction mixture was purified by mass-directed autopreparation to give the title compound.

LCMS: t_{RET}=4.39 min; MH⁺=550

### Example 25

### 2-[3-({[1-(2,4-Difluorophenyl)-6-methyl-1H-pyrazolo[3,4-d]pyrimidin-4-yl]amino}methyl)-4,4,4-trifluoro-3-hydroxy-1,1-dimethylbutyl]-4-fluorophenol

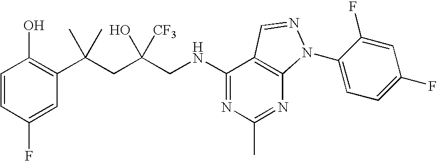

2-({[1-(2,4-Difluorophenyl)-6-methyl-1H-pyrazolo[3,4-d]pyrimidin-4-yl]amino}methyl)-1,1,1-trifluoro-4-[5-fluoro-2-(methyloxy)phenyl]-4-methyl-2-pentanol (enantiomer 1) (Example 16A, 40 mg, 0.072 mmol) was dissolved in anhydrous dichloromethane (0.15 ml) and cooled to −78° C. (cardice/acetone bath) under nitrogen. Boron tribromide (1.0M in dichloromethane) (0.36 ml, 0.36 mmol) was then added portionwise and after 5 minutes the mixture was allowed to warm to room temperature. The reaction was stirred at room temperature for 5 hours, re-cooled to −78° C. and quenched with methanol (1 ml). The reaction was warmed to room temperature, partitioned between dichloromethane (1 ml) and aqueous saturated sodium hydrogen carbonate solution (2 ml), poured onto a hydrophobic frit and the dichloromethane layer collected and evaporated under nitrogen overnight to yield crude material. The crude product was purified by mass-directed autopreparation. The appropriate fractions were neutralised using aqueous sodium hydrogen carbonate solution and extracted into dichloromethane. The organic layer was separated and the aqueous phase was back-extracted with further dichloromethane. The combined organic layers were washed with water followed by brine, filtered through a hydrophobic frit and evaporated in vacuo to give the title compound (32 mg).

LCMS: t_{RET}=3.86 min; MH⁺=540

### Example 26

### 2-{[(6-Ethyl-1-phenyl-1H-pyrazolo[3,4-d]pyrimidin-4-yl)amino]methyl}-1,1,1-trifluoro-4-[5-fluoro-2-(methyloxy)phenyl]-4-methyl-2-pentanol

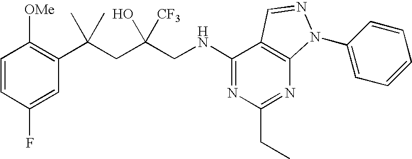

Prepared similarly to Example 2 from racemic 2-{2-[5-fluoro-2-(methyloxy)phenyl]-2-methylpropyl}-2-(trifluoromethyl)oxirane and 6-ethyl-1-phenyl-1H-pyrazolo[3,4-d]pyrimidin-4-amine. The crude reaction mixture was purified by mass-directed autopreparation to give the title compound.

LCMS: t_{RET}=4.37 min; MH⁺=532

### Example 27

### 3-(4-{[4-[5-Fluoro-2-(methyloxy)phenyl]-2-hydroxy-4-methyl-2-(trifluoromethyl)pentyl]amino}-6-methyl-1H-pyrazolo[3,4-d]pyrimidin-1-yl)benzonitrile

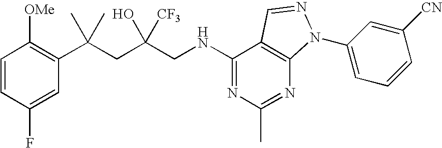

Prepared similarly to Example 2 from racemic 2-{2-[5-fluoro-2-(methyloxy)phenyl]-2-methylpropyl}-2-(trifluoromethyl)oxirane and 3-(4-amino-6-methyl-1H-pyrazolo[3,4-d]pyrimidin-1-yl)benzonitrile. The crude reaction mixture was purified by mass-directed autopreparation to give the title compound.

LCMS: t_{RET}=4.22 min; MH⁺=543

### Example 28

### 1,1,1-Trifluoro-4-[5-fluoro-2-(methyloxy)phenyl]-2-({[1-(2-fluoro-3-pyridinyl)-6-methyl-1H-pyrazolo[3,4-d]pyrimidin-4-yl]amino}methyl)-4-methyl-2-pentanol

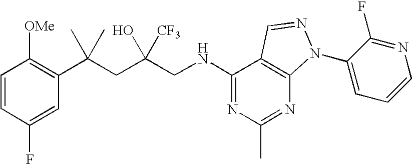

Prepared similarly to Example 2 from racemic 2-{2-[5-fluoro-2-(methyloxy)phenyl]-2-methylpropyl}-2-(trifluoromethyl)oxirane and 1-(2-fluoro-3-pyridinyl)-6-methyl-1H-pyrazolo[3,4-d]pyrimidin-4-amine. The crude reaction mixture was purified by custom purification (see below) to give the title compound.

LCMS: t_{RET}=3.79 min; MH⁺=537

| Column: | Supelcosil ABZ + plus 100 × 21.2 mm, 5 um | |
|---|---|---|
| Eluent: | Organic - 95% MeCN + 0.05% Formic Acid | |
| | Aqueous - Water + 0.1% Formic Acid | |
| | Time (mins) | % Organic |
| Gradient: | 0 | 50 |
| | 1 | 50 |
| | 25 | 80 |
| | 27 | 100 |
| | 28.5 | 100 |
| | 29 | 50 |
| | 30 | 50 |

| | |
|---|---|
| Run Time: | 30 minutes |
| Flow Rate: | 20 ml/minute |

### Example 29

### 3-(4-{[4-(2,3-Dihydro-1-benzofuran-7-yl)-2-hydroxy-4-methyl-2-(trifluoromethyl)pentyl]amino}-6-ethyl-1H-pyrazolo[3,4-d]pyrimidin-1-yl)benzonitrile

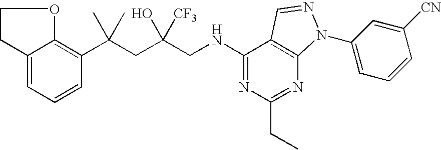

Prepared similarly to Example 2 from racemic 7-{1,1-dimethyl-2-[2-(trifluoromethyl)-2-oxiranyl]ethyl}-2,3-dihydro-1-benzofuran and 3-(4-amino-6-ethyl-1H-pyrazolo[3,4-d]pyrimidin-1-yl)benzonitrile. The crude reaction mixture was purified by mass-directed autopreparation to give the title compound.

LCMS: t_{RET}=4.36 min; MH⁺=551

### Example 30

### Methyl 3-(4-{[4-(2,3-dihydro-1-benzofuran-7-yl)-2-hydroxy-4-methyl-2-(trifluoromethyl)pentyl]amino}-6-ethyl-1H-pyrazolo[3,4-d]pyrimidin-1-yl)benzoate

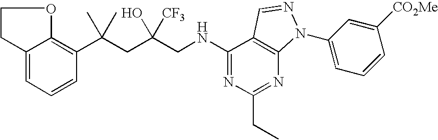

Prepared similarly to Example 2 from racemic 7-{1,1-dimethyl-2-[2-(trifluoromethyl)-2-oxiranyl]ethyl}-2,3-dihydro-1-benzofuran and methyl 3-(4-amino-6-ethyl-1H-pyrazolo[3,4-d]pyrimidin-1-yl)benzoate. The crude reaction mixture was purified by mass-directed autopreparation to give the title compound.

LCMS: t_{RET}=4.39 min; MH⁺=584

### Biological Experimental

### Glucocorticoid Receptor Binding Assay

The ability of compounds to bind to the glucocorticoid receptor was determined by assessing their ability to compete with an Alexa 555 fluorescently-labelled dexamethasone derivative. Compounds were solvated and diluted in DMSO, and transferred directly into assay plates. Fluorescent dexamethasone and a partially purified full length glucocorticoid receptor were added to the plates, together with buffer components to stabilise the GR protein (including stabilisation peptide (Panvera catalogue number P2815)) and incubated at room temperature for 2 hrs in the dark. Binding of each compound was assessed by analysing the displacement of fluorescent ligand by measuring the decrease in fluorescence polarisation signal from the mixture.

Examples 1 to 30 have glucocorticoid binding with a pIC₅₀>6.5 in this assay.

### Glucocorticoid Mediated Transrepression of NFkB Activity

Human A549 lung epithelial cells were engineered to contain a secreted placental alkaline phosphatase gene under the control of the distal region of the NFkB dependent ELAM promoter as previously described in Ray, K. P., Farrow, S., Daly, M., Talabot, F. and Searle, N. “Induction of the E-selectin promoter by interleukin 1 and tumour necrosis factor alpha, and inhibition by glucocorticoids” Biochemical Journal (1997) 328: 707-15.

Compounds were solvated and diluted in DMSO, and transferred directly into assay plates such that the final concentration of DMSO was 0.7%. Following the addition of cells (40K per well), plates were incubated for 1 hr prior to the addition of 3 ng/ml human recombinant TNFα. Following continued incubation for 16 hr, alkaline phosphatase activity was determined by measuring the change in optical density at 405 nM with time following the addition of 0.7 volumes of assay buffer (1 mg/ml p-nitrophenylphosphate dissolved in 1M diethanolamine, 0.28M NaCl, 0.5 mM MgCl₂). Dose response curves were constructed from which EC₅₀ values were estimated.

Examples 1 to 30 show pIC₅₀>7 in this assay

Examples 1 to 13, 14B to 16A, 17 to 25 and 28 show pIC₅₀>8 in this assay

### Assay for Progesterone Receptor Activity

A T225 flask of CV-1 cells at a density of 80% confluency was washed with PBS, detached from the flask using 0.25% trypsin and counted using a Sysmex KX-21N. Cells were diluted in DMEM containing 10% Hyclone, 2 mM L-Glutamate and 1% Pen/Strep at 140 cells/μl and transduced with 10% PRb-BacMam and 10% MMTV-BacMam. 70 ml of suspension cells were dispensed to each well of white Nunc 384-well plates, containing compounds at the required concentration. After 24 h 10 μl of Steady Glo were added to each well of the plates. Plates were incubated in the dark for 10 min before reading them on a Viewlux reader. Dose response curves were constructed from which pEC₅₀ values were estimated.

Examples 13 to 28 show pEC₅₀<5.5 in this assay.

In describing those examples which are preferred or more preferred according to their activity in the assays above, it will be appreciated that at least one isomer, for example, an enantiomer in a mixture of isomers (such as a racemate) has the described activity. The other enantiomer may have similar activity, less activity, no activity or may have some antagonist activity in the case of a functional assay.

Throughout the specification and the claims which follow, unless the context requires otherwise, the word ‘comprise’, and variations such as ‘comprises’ and ‘comprising’, will be understood to imply the inclusion of a stated integer or step or group of integers but not to the exclusion of any other integer or step or group of integers or steps.

The application of which this description and claims forms part may be used as a basis for priority in respect of any subsequent application. The claims of such subsequent application may be directed to any feature or combination of features described herein. They may take the form of product, composition, process, or use claims and may include, by way of example and without limitation, the following claims.

The patents and patent applications described in this application are herein incorporated by reference.

What is claimed is:

1. A compound of formula (I): 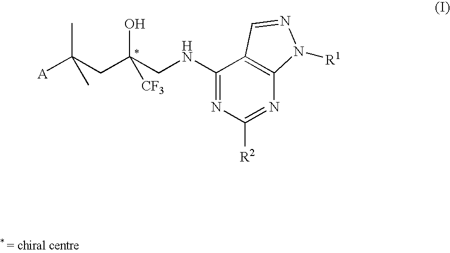
wherein
A represents 2,3-dihydro-1-benzofuran-7-yl, 5-fluoro-2-methoxy-phenyl or 5-fluoro-2-hydroxy-phenyl;
R¹ represents phenyl, pyridyl or thienyl wherein the phenyl group may be optionally substituted by one or two groups independently selected from fluorine, cyano, —C(O)OCH₃ and —C(O)OCH₂CH₃, and the pyridyl group may be optionally substituted by one fluorine group; and
R² represents methyl or ethyl;
or a physiologically functional derivative thereof.

2. A compound according to claim 1 wherein A represents 5-fluoro-2-methoxy-phenyl.

3. A compound according to claim 1 wherein R¹ represents phenyl optionally substituted by one or two fluorine groups.

4. A compound according to claim 3 wherein R¹ represents 2,4-difluorophenyl.

5. A compound according to claim 1 wherein R² represents methyl.

6. (canceled)

7. A compound which is selected from the group consisting of:
4-(2,3-dihydro-1-benzofuran-7-yl)-1,1,1-trifluoro-4-methyl-2-{[(6-methyl-1-phenyl-1H-pyrazolo[3,4-d]pyrimidin-4-yl)amino]methyl}-2-pentanol;
4-(2,3-dihydro-1-benzofuran-7-yl)-1,1,1-trifluoro-2-({[1-(2-fluorophenyl)-6-methyl-1H-pyrazolo[3,4-d]pyrimidin-4-yl]amino}methyl)-4-methyl-2-pentanol;
4-(2,3-dihydro-1-benzofuran-7-yl)-1,1,1-trifluoro-2-({[1-(4-fluorophenyl)-6-methyl-1H-pyrazolo[3,4-d]pyrimidin-4-yl]amino}methyl)-4-methyl-2-pentanol;
2-({[1-(2,4-difluorophenyl)-6-methyl-1H-pyrazolo[3,4-d]pyrimidin-4-yl]amino}methyl)-4-(2,3-dihydro-1-benzofuran-7-yl)-1,1,1-trifluoro-4-methyl-2-pentanol;
4-(2,3-dihydro-1-benzofuran-7-yl)-2-{[(6-ethyl-1-phenyl-1H-pyrazolo[3,4-d]pyrimidin-4-yl)amino]methyl}-1,1,1-trifluoro-4-methyl-2-pentanol;
4-(2,3-dihydro-1-benzofuran-7-yl)-2-({[6-ethyl-1-(2-fluorophenyl)-1H-pyrazolo[3,4-d]pyrimidin-4-yl]amino}methyl)-1,1,1-trifluoro-4-methyl-2-pentanol;
4-(2,3-dihydro-1-benzofuran-7-yl)-2-({[6-ethyl-1-(4-fluorophenyl)-1H-pyrazolo[3,4-d]pyrimidin-4-yl]amino}methyl)-1,1,1-trifluoro-4-methyl-2-pentanol;
2-({[1-(2,4-difluorophenyl)-6-ethyl-1H-pyrazolo[3,4-d]pyrimidin-4-yl]amino}methyl)-4-(2,3-dihydro-1-benzofuran-7-yl)-1,1,1-trifluoro-4-methyl-2-pentanol;
4-(2,3-dihydro-1-benzofuran-7-yl)-2-({[6-ethyl-1-(3-pyridinyl)-1H-pyrazolo[3,4-d]pyrimidin-4-yl]amino}methyl)-1,1,1-trifluoro-4-methyl-2-pentanol;
4-(2,3-dihydro-1-benzofuran-7-yl)-2-({[6-ethyl-1-(4-pyridinyl)-1H-pyrazolo[3,4-d]pyrimidin-4-yl]amino}methyl)-1,1,1-trifluoro-4-methyl-2-pentanol;
4-(2,3-dihydro-1-benzofuran-7-yl)-2-({[6-ethyl-1-(6-fluoro-3-pyridinyl)-1H-pyrazolo[3,4-d]pyrimidin-4-yl]amino}methyl)-1,1,1-trifluoro-4-methyl-2-pentanol;
4-(2,3-dihydro-1-benzofuran-7-yl)-2-({[6-ethyl-1-(3-thienyl)-1H-pyrazolo[3,4-d]pyrimidin-4-yl]amino}methyl)-1,1,1-trifluoro-4-methyl-2-pentanol;
1,1,1-trifluoro-4-[5-fluoro-2-(methyloxy)phenyl]-4-methyl-2-{[(6-methyl-1-phenyl-1H-pyrazolo[3,4-d]pyrimidin-4-yl)amino]methyl}-2-pentanol;
1,1,1-trifluoro-4-[5-fluoro-2-(methyloxy)phenyl]-2-({[1-(2-fluorophenyl)-6-methyl-1H-pyrazolo[3,4-d]pyrimidin-4-yl]amino}methyl)-4-methyl-2-pentanol;
1,1,1-trifluoro-4-[5-fluoro-2-(methyloxy)phenyl]-2-({[1-(4-fluorophenyl)-6-methyl-1H-pyrazolo[3,4-d]pyrimidin-4-yl]amino}methyl)-4-methyl-2-pentanol;
2-({[1-(2,4-difluorophenyl)-6-methyl-1H-pyrazolo[3,4-d]pyrimidin-4-yl]amino}methyl)-1,1,1-trifluoro-4-[5-fluoro-2-(methyloxy)phenyl]-4-methyl-2-pentanol;
1,1,1-trifluoro-4-[5-fluoro-2-(methyloxy)phenyl]-4-methyl-2-({[6-methyl-1-(3-pyridinyl)-1H-pyrazolo[3,4-d]pyrimidin-4-yl]amino}methyl)-2-pentanol;
1,1,1-trifluoro-4-[5-fluoro-2-(methyloxy)phenyl]-4-methyl-2-({[6-methyl-1-(4-pyridinyl)-1H-pyrazolo[3,4-d]pyrimidin-4-yl]amino}methyl)-2-pentanol;
1,1,1-trifluoro-4-[5-fluoro-2-(methyloxy)phenyl]-4-methyl-2-({[6-methyl-1-(3-thienyl)-1H-pyrazolo[3,4-d]pyrimidin-4-yl]amino}methyl)-2-pentanol;
1,1,1-trifluoro-4-[5-fluoro-2-(methyloxy)phenyl]-2-({[1-(6-fluoro-3-pyridinyl)-6-methyl-1H-pyrazolo[3,4-d]pyrimidin-4-yl]amino}methyl)-4-methyl-2-pentanol;
4-(4-{[4-[5-fluoro-2-(methyloxy)phenyl]-2-hydroxy-4-methyl-2-(trifluoromethyl)pentyl]amino}-6-methyl-1H-pyrazolo[3,4-d]pyrimidin-1-yl)benzonitrile;
ethyl 3-(4-{[4-[5-fluoro-2-(methyloxy)phenyl]-2-hydroxy-4-methyl-2-(trifluoromethyl)pentyl]amino}-6-methyl-1H-pyrazolo[3,4-d]pyrimidin-1-yl)benzoate;
methyl 3-(4-{[4-[5-fluoro-2-(methyloxy)phenyl]-2-hydroxy-4-methyl-2-(trifluoromethyl)pentyl]amino}-6-methyl-1H-pyrazolo[3,4-d]pyrimidin-1-yl)benzoate;
2-({[6-ethyl-1-(4-fluorophenyl)-1H-pyrazolo[3,4-d]pyrimidin-4-yl]amino}methyl)-1,1,1-trifluoro-4-[5-fluoro-2-(methyloxy)phenyl]-4-methyl-2-pentanol;
2-[3-({[1-(2,4-difluorophenyl)-6-methyl-1H-pyrazolo[3,4-d]pyrimidin-4-yl]amino}methyl)-4,4,4-trifluoro-3-hydroxy-1,1-dimethylbutyl]-4-fluorophenol;
2-({[6-ethyl-1-phenyl-1H-pyrazolo[3,4-d]pyrimidin-4-yl]amino}methyl)-1,1,1-trifluoro-4-[5-fluoro-2-(methyloxy)phenyl]-4-methyl-2-pentanol;
3-(4-{[4-[5-fluoro-2-(methyloxy)phenyl]-2-hydroxy-4-methyl-2-(trifluoromethyl)pentyl]amino}-6-methyl-1H-pyrazolo[3,4-d]pyrimidin-1-yl)benzonitrile;
1,1,1-trifluoro-4-[5-fluoro-2-(methyloxy)phenyl]-2-({[1-(2-fluoro-3-pyridinyl)-6-methyl-1H-pyrazolo[3,4-d]pyrimidin-4-yl]amino}methyl)-4-methyl-2-pentanol;
4-(2,3-dihydro-1-benzofuran-7-yl)-2-({[6-ethyl-1-(3-cyanophenyl)-1H-pyrazolo[3,4-d]pyrimidin-4-yl]amino}methyl)-1,1,1-trifluoro-4-methyl-2-pentanol;
methyl 3-(4-{[4-[2,3-dihydro-1-benzofuran-7-yl]-2-hydroxy-4-methyl-2-(trifluoromethyl)pentyl]amino}-6-ethyl-1H-pyrazolo[3,4-d]pyrimidin-1-yl)benzoate; and
a physiologically functional derivative thereof.

8. A compound which is selected from the group consisting of:
4-(2,3-dihydro-1-benzofuran-7-yl)-1,1,1-trifluoro-2-({[1-(2-fluorophenyl)-6-methyl-1H-pyrazolo[3,4-d]pyrimidin-4-yl]amino}methyl)-4-methyl-2-pentanol;
2-({[1-(2,4-difluorophenyl)-6-methyl-1H-pyrazolo[3,4-d]pyrimidin-4-yl]amino}methyl)-4-(2,3-dihydro-1-benzofuran-7-yl)-1,1,1-trifluoro-4-methyl-2-pentanol;
2-({[1-(2,4-difluorophenyl)-6-ethyl-1H-pyrazolo[3,4-d]pyrimidin-4-yl]amino}methyl)-4-(2,3-dihydro-1-benzofuran-7-yl)-1,1,1-trifluoro-4-methyl-2-pentanol;
1,1,1-trifluoro-4-[5-fluoro-2-(methyloxy)phenyl]-2-({[1-(2-fluorophenyl)-6-methyl-1H-pyrazolo[3,4-d]pyrimidin-4-yl]amino}methyl)-4-methyl-2-pentanol (Enantiomer 2);
2-({[1-(2,4-difluorophenyl)-6-methyl-1H-pyrazolo[3,4-d]pyrimidin-4-yl]amino}methyl)-1,1,1-trifluoro-4-[5-fluoro-2-(methyloxy)phenyl]-4-methyl-2-pentanol (Enantiomer 1);
1,1,1-trifluoro-4-[5-fluoro-2-(methyloxy)phenyl]-4-methyl-2-({[6-methyl-1-(4-pyridinyl)-1H-pyrazolo[3,4-d]pyrimidin-4-yl]amino}methyl)-2-pentanol;
2-[3-({[1-(2,4-difluorophenyl)-6-methyl-1H-pyrazolo[3,4-d]pyrimidin-4-yl]amino}methyl)-4,4,4-trifluoro-3-hydroxy-1,1-dimethylbutyl]-4-fluorophenol; and
a physiologically functional derivative thereof.

9. A compound which is selected from the group consisting of:
1,1,1-trifluoro-4-[5-fluoro-2-(methyloxy)phenyl]-2-({[1-(2-fluorophenyl)-6-methyl-1H-pyrazolo[3,4-d]pyrimidin-4-yl]amino}methyl)-4-methyl-2-pentanol (Enantiomer 2);
2-({[1-(2,4-difluorophenyl)-6-methyl-1H-pyrazolo[3,4-d]pyrimidin-4-yl]amino}methyl)-1,1,1-trifluoro-4-[5-fluoro-2-(methyloxy)phenyl]-4-methyl-2-pentanol (Enantiomer 1); and
a physiologically functional derivative thereof.

10-14. (canceled)

15. A method for the treatment of a human or animal subject with an inflammatory and/or allergic condition, which method comprises administering to said human or animal subject an effective amount of a compound as claimed in claim 1, or a physiologically functional derivative thereof.

16. A method for the treatment of a human or animal subject with skin disease selected from the group consisting of eczema, psoriasis, allergic dermatitis, neurodermatitis, pruritis and hypersensitivity reactions, which method comprises administering to said human or animal subject an effective amount of a compound as claimed in claim 1, or a physiologically functional derivative thereof.

17. A pharmaceutical composition comprising a compound of formula (I) as claimed in claim 1, or a physiologically functional derivative thereof, in admixture with one or more physiologically acceptable diluents or carriers.

18. A pharmaceutical aerosol formulation comprising a compound of formula (I) as defined in claim 1, or a physiologically functional derivative thereof, and a fluorocarbon or hydrogen-containing chlorofluorocarbon as propellant, optionally in combination with a surfactant and/or a cosolvent.

19. A pharmaceutical aerosol formulation as claimed in claim 18 wherein the propellant is selected from the group consisting of 1,1,1,2-tetrafluoroethane, 1,1,1,2,3,3,3-heptafluoro-n-propane and mixtures thereof.

20. A combination comprising a compound as claimed in claim 1, or a physiologically functional derivative thereof, and one or more other therapeutically active agents.

21. A combination according to claim 20 in which said therapeutically active agent is a β₂-adrenoreceptor agonist.

22. A combination according to claim 20 in which said therapeutically active agent is PDE4 inhibitor.

23. A process for the preparation of a compound of formula (I) as claimed in claim 1, or a physiologically functional derivative thereof, comprising reacting an epoxide of formula (II) 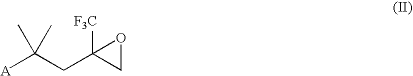
with a 4-amino-1-arylpyrazolopyrimidine of formula (III)

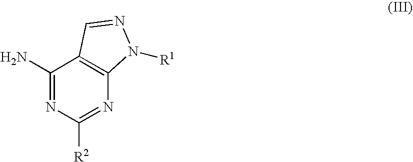

24. A compound of formula (III): 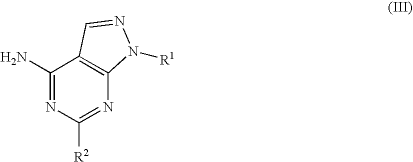
wherein R¹ represents phenyl, pyridyl or thienyl wherein the phenyl group may be optionally substituted by one or two groups independently selected from fluorine, cyano, —C(O)OCH₃ and —C(O)OCH₂CH₃ and the pyridyl group may be optionally substituted by one fluorine group; and
R² represents methyl or ethyl.

25. A process for the preparation of a compound of formula (III) as claimed in claim 24 comprising:
a) reacting a 1H-pyrazolopyrimidine-4-amine of formula (IV): 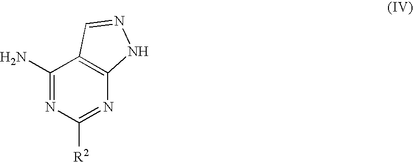
with an aryl iodide of formula (V)
I—R¹ (V)
b) reacting a 4-cyano-5-aminopyrazole of formula (VII): 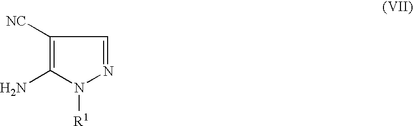
with either a nitrile of formula (VIII): 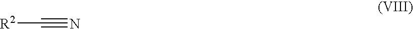
or an amidine of formula (IX):

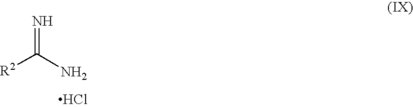

26. The method according to claim 15, wherein the method comprises administering to a human an effective amount of the compound, or a physiologically functional derivative thereof.

27. The method according to claim 16, wherein the method comprises administering to a human an effective amount of the compound, or a physiologically functional derivative thereof.
